(19) United States
(12) Patent Application Publication
(10) Pub. No.: US 2025/0161462 A1
(43) Pub. Date: May 22, 2025

(54) ENDOSOMAL CLEAVABLE HYDROPHILIC-MASKED CATIONIC CHARGE DELIVERY VEHICLES

(71) Applicant: The Regents of the University of California, Oakland, CA (US)

(72) Inventors: Steven F. Dowdy, La Jolla, CA (US); Satish G. Jadhav, San Diego, CA (US)

(21) Appl. No.: 18/841,637

(22) PCT Filed: Mar. 2, 2023
(86) PCT No.: PCT/US2023/063582

§ 371 Date: Aug. 26, 2024

Related U.S. Application Data
(60) Provisional application No. 63/316,233, filed on Mar. 3, 2022.

(51) Int. Cl. A61K 47/54; A61K 47/69; A61K 48/00

(52) U.S. Cl. CPC A61K 47/549; A61K 47/543; A61K 47/6925; A61K 48/0033

(57) ABSTRACT

The disclosure provides for compounds and compositions comprising hydrophilic-masked cationic charge dendrimers, and applications thereof, including delivering siRNA, ASO, PMO, PNA, oligonucleotide and nucleic acid vectors. The methods and approaches disclosed herein can also be applied to lipids to make hydrophilic-masked cationic charge lipid nanoparticles for mRNA, RNA, siRNA, DNA, ASO, PMO, PNA, oligonucleotide and nucleic acid vector delivery.

### CROSS REFERENCE TO RELATED APPLICATIONS

The application claims priority under 35 U.S.C. § 119 to U.S. Provisional Application Ser. No. 63/316,233, filed March 3, 2022, the disclosures of which are incorporated herein by reference.

STATEMENT REGARDING FEDERALLY SPONSORED RESEARCH

This invention was made with government support under Grant Nos. CA267883 and Grant No. NS116663 awarded by the National Institutes of Health. The government has certain rights in the invention.

### TECHNICAL FIELD

The disclosure provides for compounds and compositions comprising hydrophilic-masked cationic charge dendrimers, and applications thereof, including delivering siRNA, ASO, PMO, PNA and other derivatized oligonucleotides and nucleic acid vectors. The methods and approaches disclosed herein can also be applied to lipids to make hydrophilic-masked cationic charge lipid nanoparticles for siRNA, ASO, and nucleic acid vector delivery.

### BACKGROUND

Eukaryotic cells contain several thousand proteins, which have been, during the course of evolution, selected to play specific roles in the maintenance of virtually all cellular functions. Not surprisingly, the viability of every cell, as well as the organism on the whole, is intimately dependent on the correct expression of these proteins. Factors which affect a particular protein's function, either by mutations or deletions in the amino acid sequence, or through changes in expression to cause over-expression or suppression of protein levels, invariably lead to alterations in normal cellular function. Such alterations often directly underlie a wide variety of genetic and acquired disorders. Consequently, the ability to target and selectively inhibit, alter or increase expression of genes or kill cells comprising mutations that result in cell proliferative disorders would help to control such diseases and disorders.

In practice, however, the direct intracellular delivery of these agents has been difficult. This is due primarily to the bioavailability barrier of the cell's lipid bilayer plasma membrane, which effectively prevents the uptake of the majority of peptides, proteins, RNAs, oligonucleotides, DNAs, CRISPR and other agents by limiting their passive entry. Even if the agent is able to be taken up by a cell through endosomal uptake mechanisms, due to the endosomal lipid bilayer membrane, the release of the agent intracellularly is still a rate limiting step.

### SUMMARY

The disclosure provides methods and compositions useful for delivery of molecules into cells. The disclosure provides compositions of glycoside-masked dendrimers that once inside the endo/lysosomal system are cleaved by glycosidases to expose cationic charges that facilitate endosomal escape. The methods and approaches disclosed herein can be applied directly to a wide variety of oligonucleotides and to lipids used to make LNPs for mRNA and DNA vector delivery.

The disclosure provides a delivery vehicle (“construct”) for delivery of biological agents, small molecules and the like into a cell. The construct comprising (i) at least one cargo domain; (ii) a dendrimer domain having a first end and a plurality of second ends; (iii) a plurality of hydrophobic or cationic charge domains; wherein the first end of the dendrimer domain is linked directly or indirectly to the at least one cargo domain and the plurality of second ends are linked to the plurality of hydrophobic or cationic charge domains. In one embodiment, the construct further comprises a targeting domain. In another or further embodiment, the plurality of cationic charge or hydrophobic domains each comprise a hydrophilic mask. In still another or further embodiment, the plurality of cationic charge or hydrophilic domains are masked by a plurality of hydrophilic mask domains such that the construct has a reduced net cationic, a net neutral or a net anionic charge. In another or further embodiment, the dendrimer comprises 1, 2, 3, 4, or 5 generations. In another embodiment, the construct further comprises a linker or a coupler domain between the at least one cargo domain and the first end of the dendrimer domain. In still another or further embodiment of any of the foregoing, the hydrophilic mask comprises a glycoside moiety. In still another embodiment or further embodiment of any of the foregoing the hydrophobic domain or the cationic charge domain is any functional group which contains an aromatic indole ring; nitrogen containing mono-cyclic or multi-cyclic rings; primary, secondary or tertiary amino group; a lipid or a monomeric unit derived therefrom; a tocopherol; a hydrophobic oligomer or a monomeric unit derived therefrom; a hydrophobic polymer or a monomeric unit derived therefrom. In a further embodiment, the hydrophobic domain comprises a lipid selected from a C8, C10, C12, C14, C16, or C18 lipid or derivative thereof. In still another or further embodiment, the hydrophobic domain comprises a monomeric unit derived from a lipid selected from a fatty acid, a fatty alcohol and any other lipid molecule with at least two carbon units. In still another or further embodiment, the hydrophobic domain comprises a hydrophobic polymer. In another embodiment, the hydrophobic domain comprises one or more monomeric units derived from a hydrophobic polymer selected from the group consisting of: polyester, polyether, polycarbonate, polyanhydride, polyamide, polyacrylate, polymethacrylate, polyacrylamide, polysulfone, polyalkane, polyalkene, polyalkyne, polyanhydride, polyorthoester, N-isopropylacrylamide, methyl acrylate, ethyl acrylate, propyl acrylate, butyl acrylate, methyl methacrylate, ethyl methacrylate, propyl methacrylate, butyl methacrylate, acrylic acid, methacrylic acid, quaternary ammonium-modified acrylate, quaternary ammonium modified-methacrylate, acrylamide, caprolactone, lactide, and valeronolactone. In still another embodiment, the hydrophobic domain or the cationic charge domain comprises a 1H-indole group; a nitrogen containing mono- or multicyclic aromatic or non-aromatic compounds. In another embodiment, the cationic charge domain comprises a primary amine, a secondary amine, a tertiary amine, a quaternary amine, a complex amino group, or an ionizable amines. In a further embodiment, the cationic charge domain comprises metformin group, a morpholine group, a piperazine group, a pyridine group, a pyrrolidine group, piperidine, a thiomorpholine, a thiomorpholine oxide, a thiomorpholine dioxide, imidazole, guanidine group or creatine. In still a further embodiment, the cationic charge domain comprises a quartenary amine. In another embodiment, the cationic charge domain comprises spermine or spermidine or other polymeric cationic molecules. In another or further embodiment, of any of the foregoing, the at least one cargo domain comprises: RNA, DNA, ASO, PMO, PNA, mRNA, DNA vectors, oligonucleotide derivatives and/or combinations with any of these cargos as a single stranded and/or double stranded molecule. In another or further embodiment of any of the foregoing, the at least one cargo domain is selected from the group consisting of a small molecule therapeutic, a peptide, a protein, a single stranded nucleic acid, a double stranded nucleic acid, oligonucleotide and derivatives, and a protein-nucleic acid complex. In another embodiment, the dendrimer comprises 3, 4, 5, 6, 7, 8, 9, 10, 11, 12, 13, 14, 15, 16, 17, 18, 19, 20, 21, 23, 24, 25, 26, 27, 28, 29, 30, 31, 32, 33, 34, 35, 36, 37, 38, 39, 40, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 second ends, or a range that includes or is between any two of the foregoing numbers. In still another embodiment, the plurality of second ends of the dendrimer comprise spermine groups, wherein nitrogen containing groups of spermine are masked with hydrophilic masking groups. In still another embodiment, the plurality of second ends of the dendrimer comprise cationic polymers with varying numbers of cationic amines masked with hydrophilic masking groups. In still another embodiment, the at least one cargo domain is linked to the first end of the dendrimer by a covalent bond, by hydrogen bonds, or by electrostatic attraction. In another embodiment, the at least one cargo domain is linked to the first end of the dendrimer by a linker domain. In still another embodiment, the at least one cargo domain is linked to the dendrimer by an amide bond. In any of the foregoing embodiments, the hydrophilic masking groups are configured to be removed by esterases, glycosidases, lipases, proteases, phospholipases, phosphatases, sulfatases or the low pH in endosomes or lysosomes. In a further embodiment, the hydrophilic masking groups are α or β glycoside containing groups that are configured to be removed by the corresponding α or β glycosidase in endosomes or lysosomes. In a further embodiment, the glycoside is a monomer, dimer, trimer or multimeric chain of an α or β anomeric carbon configuration of glucose, mannose, galactose, N-acetylglucosamine, N-acetylgalactosamine, xylose, ribose, fucose, glucuronic acid, iduronic acid or sialic acids that is selectively cleaved inside endosomes or lysosomes by the corresponding α or β glycosidase.

The disclosure also provides a hydrophilic masked cationic charged dendrimer comprising: a dendrimer linked or complexed to cargo molecule(s); hydrophilic masking groups linked to nitrogen containing groups on the termini of the dendrimer; wherein the hydrophilic masking groups are configured to be removed by the action of enzymes or low pH found in lysosomes and/or endosomes, wherein removal of the hydrophilic masking groups reveals the nitrogen containing groups that have cationic charges, and wherein the cationic charged nitrogen containing groups of the dendrimer are configured to disrupt the membrane of lysosomes and/or endosomes leading to the intracellular release of the cargo molecule(s). In another embodiment, the dendrimer is dimer, trimer, tetramer, pentamer, hexamer, heptamer, octamer, nonamer, or decamer. In another or further embodiment, the dendrimer comprises 6, 7, 8, 9, 10, 11, 12, 13, 14, 15, 16, 17, 18, 19, 20, 21, 23, 24, 25, 26, 27, 28, 29, 30, 31, 32, 33, 34, 35, 36, 37, 38, 39, 40, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 termini, or a range of termini that includes or is between any two of the foregoing numbers. In a further embodiment, the dendrimer comprises 9, 18, 27, 36, 45, 54, 63, 72, 81, or 90 termini. In still another or further embodiment of any of the foregoing embodiments, the termini of the dendrimer comprise spermine groups, wherein the nitrogen containing groups of spermine are masked with hydrophilic masking groups. In still another or further embodiment of any of the foregoing, the nitrogen containing groups comprise a primary amine, a secondary amine, a tertiary amine, a quaternary amine, a complex amino group, or an ionizable amines. In yet another or further embodiment of any of the foregoing, the dendrimer comprises 1, 2, 3, 4, 5, 6, 7, or 8 generations of branch points, or a range of branch points that includes or is between any two of the foregoing numbers. In still yet another or further embodiment, the cargo molecule is selected from the group consisting of a small molecule therapeutic, a peptide, a protein, a single stranded nucleic acid, a double stranded nucleic acid, oligonucleotide and derivatives, and a protein-nucleic acid complex. In a further embodiment, the cargo includes: RNA, DNA, ASO, PMO, PNA, mRNA, DNA vectors, oligonucleotide derivatives and/or combinations with any of these cargos as a single stranded and/or double stranded molecule. In still yet another or further embodiment, the cargo molecule is linked to the dendrimer by a covalent bond, by hydrogen bonds, or by electrostatic attraction. In another embodiment, the cargo molecule is linked to the dendrimer by an amide bond. In still another or further embodiment of any of the foregoing embodiments, the hydrophilic masking groups are configured to be removed by esterases, glycosidases, lipases, proteases, phospholipases, phosphatases, sulfatases or the low pH in endosomes or lysosomes. In still further embodiments of any of the foregoing, the hydrophilic masking groups are α or β glycoside containing groups that are configured to be removed by the corresponding α or β glycosidase in endosomes or lysosomes. In a further embodiment, the glycoside is a monomer, dimer, trimer or multimeric chain of an α or β anomeric carbon configuration of glucose, mannose, galactose, N-acetylglucosamine, N-acetylgalactosamine, xylose, ribose, fucose, glucuronic acid, iduronic acid or sialic acids that is selectively cleaved inside endosomes or lysosomes by the corresponding α or β glycosidase.

The disclosure also provides a method to deliver cargo molecule(s) intracellularly, comprising contacting a cell with the construct or hydrophilic masked cationic charged dendrimer as set forth in the preceding paragraphs and as further defined herein. In one embodiment, the cargo molecules are delivered to the cytoplasm of the cell. In another or further embodiment, the cargo molecules are selected from the group consisting of a small molecule therapeutic, a peptide, a protein, a single stranded nucleic acid, a double stranded nucleic acid, and a protein-nucleic acid complex, RNA, DNA, ASO, PMO, PNA, mRNA, DNA vectors, oligonucleotide derivatives and/or combinations with any of these cargos as a single stranded and/or double stranded molecule. In still another or further embodiment, the cell is a cancer cell.

The disclosure also provides a hydrophilic masked cationic charged lipid nanoparticle (LNP) comprising a lipid nanoparticle comprises or is loaded with cargo molecule(s); wherein the lipid nanoparticle comprises lipids that have nitrogen containing head groups that are linked to hydrophilic masking groups; wherein the hydrophilic masking groups are configured to be removed by the action of enzymes found in lysosomes and/or endosomes, wherein removal of the hydrophilic masking groups reveals the nitrogen containing head groups of the lipids that are ionizable or have cationic charges, and wherein the ionizable or cationic charged head groups of the lipids are configured to disrupt the membrane of lysosomes and/or endosomes leading to the intracellular release of the cargo molecule(s). In one embodiment, the cargo molecules are selected from the group consisting of a small molecule therapeutic, a peptide, a protein, a single stranded nucleic acid, a double stranded nucleic acid, and a protein-nucleic acid complex. In another or further embodiment, the nitrogen containing head groups of the lipids comprise a primary amine, a secondary amine, a tertiary amine, a quaternary amine, a complex amino group, or an ionizable amines. In still another or further embodiment, the hydrophilic masking groups linked to the nitrogen containing head groups of the lipids are zwitterions. In another embodiment or further embodiment, the removal of the hydrophilic masking groups provides for ionizable head groups or cationic head groups. In still another or further embodiment, the lipids have head groups that comprise spermine. In yet another or further embodiment, the hydrophilic masking groups are configured to be removed by esterases, glycosidases, lipases, proteases, phospholipases, phosphatases or sulfatases. In still another or further embodiment, the hydrophilic masking groups are glycoside containing groups that are configured to be removed by glycosidases. In a further embodiment, the glycosidase is a monomer, dimer, trimer or multimeric chain of an α or β anomeric carbon configuration of glucose, mannose, galactose, N-acetylglucosamine, N-acetylgalactosamine, xylose, ribose, fucose, glucuronic acid, iduronic acid, sialic acids that is selectively cleaved inside endosomes or lysosomes by the corresponding α or β glycosidase.

The disclosure also provides a method to deliver cargo molecule(s) intracellularly, comprising contacting a cell with the hydrophilic masked cationic charged LNP as set forth in the prior paragraph or as further described herein. In one embodiment, the cargo molecules are delivered to the cytoplasm of the cell. In still another or further embodiment, the cargo molecules are selected from the group consisting of a small molecule therapeutic, a peptide, a protein, a single stranded nucleic acid, a double stranded nucleic acid, and a protein-nucleic acid complex, RNA, DNA, ASO, PMO, PNA, mRNA, DNA vectors, oligonucleotide derivatives and/or combinations with any of these cargos as a single stranded and/or double stranded molecule. In still another or further embodiment, the cell is a cancer cell.

The details of one or more embodiments are set forth in the accompanying drawings and the description below. Other features, objects, and advantages will be apparent from the description and drawings, and from the claims.

### BRIEF DESCRIPTION OF THE FIGURES

FIG. 1A-C provides an example of delivering an siRNA, ASO, PMO, PNA intracellularly using (A) 1x GlcA-Spermine (4 GlcA masked cationic charges); (B) 3x GlcA-Spermine (12 GlcA masked cationic charges); and (C) 9x GlcA-Spermine (SP9) (36 GlcA masked cationic charges.

FIG. 2 provides a scheme for the synthesis of mono-GlcA-Spermine BCN.

FIG. 3 provides a synthesis route of a GlcA-SP9 dendrimer of GlcA-Masked Spermine uEED. 9x GlcA-Spermine=36 GlcA masked cationic charges.

FIG. 4 provides an example of delivering an siRNA, ASO, PMO, PNA intracellularly using a GlcA-SP18 of a dimer dendrimer of a GlcA-Masked Spermine uEED. 18x GlcA-Spermine (SP9)-72 GlcA masked cationic charges.

FIG. 5 provides a scheme for the synthesis of bis-GlcA-Spermine BCN and a synthesis route of GlcA-SP18 dendrimer of GlcA-Masked Spermine ASO-uEED. 18x GlcA-Spermine=72 GlcA masked cationic charges.

FIG. 6 provides an example of delivering an siRNA, ASO, PMO, PNA intracellularly using a GlcA-SP27 of a dimer dendrimer of a GlcA-Masked Spermine uEED. 27x GlcA-Spermine (SP9)=108 GlcA masked cationic charges.

FIG. 7 provides a scheme for the synthesis of tris-GlcA-Spermine BCN and a synthesis route of GlcA-SP27 dendrimer of GlcA-Masked Spermine ASO-uEED. 27x GlcA-Spermine=108 GlcA masked cationic charges.

FIG. 8 provides an example of delivering an siRNA, ASO, PMO, PNA intracellularly using a GlcA-SP81 of a dimer dendrimer of a GlcA-Masked Spermine uEED. 81x GlcA-Spermine (SP9)=324 GlcA masked cationic charges.

FIG. 9 provides a scheme for the synthesis of nona-GlcA-Spermine BCN and a synthesis route of GlcA-SP81 dendrimer of GlcA-Masked Spermine uEED. 81x GlcA-Spermine=324 GlcA masked cationic charges.

FIG. 10 provides a general outline of glycoside-masked lipids to make LNPs.

FIG. 11 provides an example of zwitterion GlcA-SM102 LNP that masks ionizable head groups.

FIG. 12 provides a synthesis route of zwitterion GlcA-SM102 LNP that masks ionizable head groups.

FIG. 13 provides an example of zwitterion GlcA-ALC0315 LNP that masks ionizable head groups.

FIG. 14 provides a synthesis route of zwitterion GlcA-ALC0315 LNP that masks ionizable head groups.

FIG. 15 provides an example of zwitterion GlcA-MC3-like LNP that masks ionizable head groups.

FIG. 16 provides a synthesis route of zwitterion GlcA-MC3-like LNP that masks ionizable head groups.

FIG. 17 provides an example of GlcA-MC3-like LNP that masks cationic head groups of lipids.

FIG. 18 provides a synthesis route of GlcA-MC3-like LNP that masks cationic head groups of lipids.

FIG. 19 provides an example of GlcA-Sperm-SM102-like LNP that masks cationic head groups of lipids.

FIG. 20 provides a synthesis route of GlcA-Sperm-SM102-like LNP that masks cationic head groups of lipids.

FIG. 21 provides an example of GlcA-Branched Sperm-SM102-like LNP.

FIG. 22 provides a synthesis route of GlcA-Branched Sperm-SM102-like LNP.

FIG. 23A-C shows (A) the Cu-less Click conjugation of 1x GlcA-Spermine (SP1)(“uEED1”)-BCN to the 3′ end Azide of a trisGalNAc-PMO and analysis on a 15% gel after hydridization to a corresponding anionic DNA oligo. (B) the molecular weight of the trisGalNAc-PMO 1x GlcA-Spermine (SP1)(“uEED1”) by MALDI-TOF mass spectrometry. (C) HPLC profile of the trisGalNAc-PMO 1x GlcA-Spermine (SP1)(“uEED1”).

FIG. 24A-C shows (A) the Cu-less Click conjugation of 3x GlcA-Spermine (SP1)(“uEED3”)-BCN to the 3′ end Azide of a trisGalNAc-PMO and analysis on a 15% gel after hydridization to a corresponding anionic DNA oligo. (B) the molecular weight of the trisGalNAc-PMO 3x GlcA-Spermine (SP1)(“uEED3”) by MALDI-TOF mass spectrometry. (C) HPLC profile of the trisGalNAc-PMO 3x GlcA-Spermine (SP1)(“uEED3”).

FIG. 25A shows the structure of the 3x GlcA-Spermine (SP1) (“TSG”)-distearoyl glycerol lipid (TSG-DSG) before and after cleavage by Glucuronidase.

FIG. 25B shows the activity of TSG-DSG LNPs loaded with Luciferase mRNA in mouse models plus GalNAc targeting DSG lipid. Mice were injected with LNPs loaded with Luciferase mRNA or control PBS, then administered luciferin at 6 hr and whole animal IVIS imaged for luminescence (photons/sec). LNPs were composed of Luciferase mRNA (Trilink, CA) plus 50% ALC-0315 lipid, 33.5% Cholesterol, 10% phosphocholine, 5% TSG-DSG, 1% PEG-DMG and +/−0.5% GalNAc-DSG lipid. Bar graphs represent individual treated mice.

FIG. 26A shows the structure of the Glc-ALC-0315 lipid (Glc-lipid) before and after cleavage by Glucuronidase.

FIG. 26B shows the activity of Glc-lipid LNPs loaded with Luciferase mRNA in mouse models. Mice were injected with LNPs loaded with Luciferase mRNA or control PBS, then administered luciferin at 6 hr and livers were dissected and IVIS imaged for luminescence (photons/sec). LNPs were composed of Luciferase mRNA (Trilink, CA) plus 25% Glc-lipid, 25% ALC-0315 lipid, 39% Cholesterol, 10% phosphocholine, and 1% PEG-DMG. Bar graphs represent individual treated mice.

### DETAILED DESCRIPTION

As used herein and in the appended claims, the singular forms “a,” “an,” and “the” include plural referents unless the context clearly dictates otherwise. Thus, for example, reference to “a cargo” includes a plurality of such cargoes and reference to “the linker” includes reference to one or more linkers, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The publications discussed above and throughout the text are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

The term “alkenyl”, refers to an organic group that is comprised of carbon and hydrogen atoms that contains at least one double covalent bond between two carbons. Typically, an “alkenyl” as used in this disclosure, refers to organic group that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C₂-alkenyl can form a double bond to a carbon of a parent chain, an alkenyl group of three or more carbons can contain more than one double bond. It certain instances the alkenyl group will be conjugated, in other cases an alkenyl group will not be conjugated, and yet other cases the alkenyl group may have stretches of conjugation and stretches of nonconjugation. Additionally, if there is more than 2 carbon, the carbons may be connected in a linear manner, or alternatively if there are more than 3 carbons then the carbons may also be linked in a branched fashion so that the parent chain contains one or more secondary, tertiary, or quaternary carbons. An alkenyl may be substituted or unsubstituted, unless stated otherwise.

The term “alkyl”, refers to an organic group that is comprised of carbon and hydrogen atoms that contains single covalent bonds between carbons. Typically, an “alkyl” as used in this disclosure, refers to an organic group that contains 1, 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. Where if there is more than 1 carbon, the carbons may be connected in a linear manner, or alternatively if there are more than 2 carbons then the carbons may also be linked in a branched fashion so that the parent chain contains one or more secondary, tertiary, or quaternary carbons. An alkyl may be substituted or unsubstituted, unless stated otherwise.

The term “alkynyl”, refers to an organic group that is comprised of carbon and hydrogen atoms that contains a triple covalent bond between two carbons. Typically, an “alkynyl” as used in this disclosure, refers to organic group that contains that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C₂-alkynyl can form a triple bond to a carbon of a parent chain, an alkynyl group of three or more carbons can contain more than one triple bond. Where if there is more than 3 carbon, the carbons may be connected in a linear manner, or alternatively if there are more than 4 carbons then the carbons may also be linked in a branched fashion so that the parent chain contains one or more secondary, tertiary, or quaternary carbons. An alkynyl may be substituted or unsubstituted, unless stated otherwise.

The term “amino,” as used herein, represents —N(R^{N1})₂ or —N(═NR^{N1})(NR^{N1})₂ wherein each R^{N1} is, independently, H, OH, NO₂, N(R^{N2})₂, SO₂OR^{N2}, SO₂R^{N2}, SOR^{N2}, an N-protecting group, alkyl, alkenyl, alkynyl, alkoxy, aryl, alkaryl, cycloalkyl, alkcycloalkyl, heterocyclyl (e.g., heteroaryl), alkheterocyclyl (e.g., alkheteroaryl), or two R^{N1} combine to form a heterocyclyl, and wherein each R^{N2} is, independently, H, alkyl, or aryl. In one embodiment, amino is —NH₂, or —NHR^{N1}, wherein R^{N1} is, independently, OH, NO₂, NH₂, NR^{N2}₂, SO₂OR^{N2}, SO₂R^{N2}, SOR^{N2}, alkyl, or aryl, and each RN2 can be H, alkyl, or aryl. The R^{N1} groups may themselves be unsubstituted or substituted as described herein.

The term “aryl”, as used in this disclosure, refers to a conjugated planar ring system with delocalized pi electron clouds that contain only carbon as ring atoms. An “aryl” for the purposes of this disclosure encompass from 1 to 4 aryl rings wherein when the aryl is greater than 1 ring the aryl rings are joined so that they are linked, fused, or a combination thereof. An aryl may be substituted or unsubstituted, or in the case of more than one aryl ring, one or more rings may be unsubstituted, one or more rings may be substituted, or a combination thereof.

The term generally represented by the notation “Cₓ-C_{y}” (where x and y are whole integers and y>x) prior to a functional group, e.g., “C₁-C₁₂ alkyl” refers to a number range of carbon atoms. For the purposes of this disclosure any range specified by “Cₓ-C_{y}” (where x and y are whole integers and y>x) is not exclusive to the expressed range, but is inclusive of all possible ranges that include and fall within the range specified by “Cₓ-C_{y}” (where x and y are whole integers and y>x). For example, the term “C₁-C₄” provides express support for a range of 1 to 4 carbon atoms, but further provides implicit support for ranges encompassed by 1 to 4 carbon atoms, such as 1 to 2 carbon atoms, 1 to 3 carbon atoms, 2 to 3 carbon atoms, 2 to 4 carbon atoms, and 3 to 4 carbon atoms.

As used herein, “cationic polymers” refer to long chain chemical constructs that comprise positively charged side groups. Examples of positively charged polymers include poly(ethylene imine) (PEI), spermine, spermidine, and poly(amidoamine) (PAMAM).

The term “cycloalkenyl”, as used in this disclosure, refers to an alkene that contains at least 4 carbon atoms but no more than 12 carbon atoms connected so that it forms a ring. A “cycloalkenyl” for the purposes of this disclosure encompasses from 1 to 4 cycloalkenyl rings, wherein when the cycloalkenyl is greater than 1 ring, then the cycloalkenyl rings are joined so that they are linked, fused, or a combination thereof. A cycloalkenyl may be substituted or unsubstituted, or in the case of more than one cycloalkenyl ring, one or more rings may be unsubstituted, one or more rings may be substituted, or a combination thereof.

The term “cycloalkyl”, as used in this disclosure, refers to an alkyl that contains at least 3 carbon atoms but no more than 12 carbon atoms connected so that it forms a ring. A “cycloalkyl” for the purposes of this disclosure encompasses from 1 to 4 cycloalkyl rings, wherein when the cycloalkyl is greater than 1 ring, then the cycloalkyl rings are joined so that they are linked, fused, or a combination thereof. A cycloalkyl may be substituted or unsubstituted, or in the case of more than one cycloalkyl ring, one or more rings may be unsubstituted, one or more rings may be substituted, or a combination thereof.

As used herein a “dendrimer” refers to a branched scaffold structure with functionalized terminal ends for conjugating a cationic polymer or cationic molecule. Dendrimers suitable for use include, but are not limited to, polyamidoamine (PAMAM), polypropylamine (POPAM), polyethylenimine, polylysine, polyester, iptycene, aliphatic poly(ether), and/or aromatic polyether dendrimers. Each dendrimer of the dendrimer complex may be of similar or different chemical nature than the other dendrimers (e.g., the first dendrimer may include a PAMAM dendrimer, while the second dendrimer may comprise a POPAM dendrimer).

A dendrimer of the disclosure can include multiple dendrimers. For example, the dendrimer complex can include a third dendrimer; wherein the third dendrimer is complexed with at least one other dendrimer. In another embodiment, the first and second dendrimers are each complexed to a third dendrimer, wherein the first and second dendrimers are PAMAM dendrimers and the third dendrimer is a POPAM dendrimer.

The dendrimers for use in the disclosure include unsymmetrical or asymmetrical dendrimers having more than one radius due to asymmetry of the dendrimer. In some embodiments, the asymmetrical dendrimer has two different radii. Such dendrimers and the synthesis thereof are further described in Lee et al., Bioconjugate Chem. 18: 579-584 (2007).

The disclosure contemplates the use of any type of dendrimer including but not limited to poly(amidoamine) (PAMAM) dendrimers such as dense star polymers and Starburst polymers, poly(amidoamine-organosilicon) (PAMAMOS) dendrimers, (Poly(Propylene Imine)) (PPI) dendrimers, tecto dendrimers, multilingual dendrimers, chiral dendrimers, hybrid dendrimers/linear polymers, amphiphilic dendrimers, micellar dendrimers and Fréchet-type dendrimers.

In one embodiment, the dendrimer conjugate comprises a PAMAM dendrimer. PAMAM dendrimers are a family of water-soluble polymers characterized by a unique tree-like branching architecture and a compact spherical shape in solution. Several classes of PAMAM dendrimers have been synthesized using different cores such as ethylene diamine (EDA) and 1,4-diamino butane (DAB) with different surface groups (e.g. amine, hydroxyl, or carboxyl). PAMAM dendrimers are identified by a generation number (Gn) in the range 0-10 where an increase in Gn denotes a controlled incremental increase in size, molecular weight, and number of surface groups. PAMAM dendrimers are efficient drug carriers due to the high degree of branching and the large number of surface groups, which can be utilized to immobilize drugs, imaging agents, or targeting ligands to achieve a high density of therapeutic molecules in a compact system.

PAMAMOS dendrimers are composed of radially layered poly(amidoamine-organosilicon) units. These dendrimers are inverted unimolecular micelles that consist of hydrophilic, nucleophilic PAMAM interiors and hydrophobic organosilicon (OS) exteriors. These dendrimers may serve as precursors for the preparation of honeycomb-like networks with nanoscopic PAMAM and OS domains.

PPI dendrimers are generally poly-alkyl amines having primary amines as terminal groups. The PPI dendrimer interior consists of numerous tertiary tris-propylene amines. PPI dendrimers are also known as POPAM (Poly(Propylene Amine) with DAB cores.

Tecto dendrimers are composed of a core dendrimer, surrounded by dendrimers of differing type in order to impart specific regional functionality. Multilingual dendrimers are dendrimers in which the surface contains multiple copies of a particular functional group. Chiral dendrimers are based upon the construction of constitutionally different but chemically similar branches to chiral core. Hybrid dendrimers/linear polymers are hybrids (block or graft polymers) of dendritic and linear polymers. Amphiphilic dendrimers are dendrimers that have two segregated sites of chain end, one half is electron donating and the other half is electron withdrawing. Micellar dendrimers are unimolecular micelles of water soluble hyper branched polyphenylenes.

Fréchet-Type dendrimers are based on a poly-benzyl ether hyper-branched skeleton. These dendrimers usually have carboxylic acid groups as surface groups, serving as a good anchoring point for further surface fictionalization, and as polar surface groups to increase the solubility of this hydrophobic dendrimer type in polar solvents or aqueous media.

The term “disorder” as used herein is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

The term “glycoside” refers to a molecule in which a sugar group is bonded through its anomeric carbon to another group via a glycosidic bond. Glycosides can be linked by an O— (an O-glycoside), N— (a glycosylamine), S— (a thioglycoside), or C— (a C-glycoside) glycosidic bond. An empirical formula is Cₘ(H₂O)ₙ (where m can be different from n, and m and n are <36), Glycoside herein includes a monomer, dimer, trimer or multimeric chain of glucose (dextrose), fructose (levulose) allose, altrose, mannose, gulose, iodose, galactose, talose, galactosamine, glucosamine, sialic acid, N-acetylglucosamine, sulfoquinovose (6-deoxy-6-sulfo-D-glucopyranose), N-acetylgalactosamine, ribose, fucose, arabinose, xylose, lyxose, sorbitol, mannitol, sucrose, lactose, maltose, trehalose, maltodextrins, raffinose, Glucuronic acid (glucuronide), iduronic acid, sialic acids and stachyose. It can be in D form or L form, 5 atoms cyclic furanose forms, 6 atoms cyclic pyranose forms, or acyclic form, α-isomer (the ——OH of the anomeric carbon below the plane of the carbon atoms of Haworth projection), or a β-isomer (the ——OH of the anomeric carbon above the plane of Haworth projection). It is used herein as a monosaccharide, disaccharide, polyols, or oligosaccharides containing 3-6 sugar units. Of particular use in the compositions and methods of the disclosure are glycosides that can be cleaved by endosomal glycosidases. Glycosidases (sometimes referred to as glycoside hydrolases) are known enzymes that hydrolyze glycosidic bonds. Glycosidases are classified in EC 3.2.1 as enzymes that catalyze the hydrolysis of O- or S-glycosides.

The term “hydrophilic group,” or “hydrophilic domain,” as used herein, represents a moiety or domain that confers an affinity to water and increase the solubility of a construct in water. Hydrophilic groups can be ionic or non-ionic and include moieties that are positively charged, negatively charged, and/or can engage in hydrogen-bonding interactions.

The term “hetero-” when used as a prefix, such as, hetero-alkyl, hetero-alkenyl, hetero-alkynyl, or hetero-hydrocarbon, for the purpose of this disclosure refers to the specified hydrocarbon having one or more carbon atoms replaced by non-carbon atoms as part of the parent chain. Examples of such non-carbon atoms include, but are not limited to, N, O, S, Si, Al, B, and P. If there is more than one non-carbon atom in the hetero-based parent chain then this atom may be the same element or may be a combination of different elements, such as N and O. In a particular embodiment, a “hetero”-hydrocarbon (e.g., alkyl, alkenyl, alkynyl) refers to a hydrocarbon that has from 1 to 3 C, N and/or S atoms as part of the parent chain.

The term “heterocycle,” as used herein, refers to ring structures that contain at least 1 noncarbon ring atom. A “heterocycle” for the purposes of this disclosure encompass from 1 to 4 heterocycle rings, wherein when the heterocycle is greater than 1 ring the heterocycle rings are joined so that they are linked, fused, or a combination thereof. A heterocycle may be aromatic or nonaromatic, or in the case of more than one heterocycle ring, one or more rings may be nonaromatic, one or more rings may be aromatic, or a combination thereof. A heterocycle may be substituted or unsubstituted, or in the case of more than one heterocycle ring one or more rings may be unsubstituted, one or more rings may be substituted, or a combination thereof. Typically, the noncarbon ring atom is N, O, S, Si, Al, B, or P. In the case where there is more than one noncarbon ring atom, these noncarbon ring atoms can either be the same element, or combination of different elements, such as N and O. Examples of heterocycles include, but are not limited to: a monocyclic heterocycle such as, aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide; and polycyclic heterocycles such as, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine. In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2. 1]heptane.

The terms “heterocyclic group”, “heterocyclic moiety”, “heterocyclic”, or “heterocyclo” used alone or as a suffix or prefix, refers to a heterocycle that has had one or more hydrogens removed therefrom.

The term “hydrocarbons” refers to groups of atoms that contain only carbon and hydrogen. Examples of hydrocarbons that can be used in this disclosure include, but are not limited to, alkanes, alkenes, alkynes, arenes, and benzyls.

The term “hydrophilic group,” or “hydrophilic domain,” as used herein, represents a moiety or domain that confers an affinity to water and increase the solubility of an construct in water. Hydrophilic groups can be ionic or non-ionic and include moieties that are positively charged, negatively charged, and/or can engage in hydrogen-bonding interactions.

As used herein a “ionizable lipid” or “cationic lipid” refers to a lipid that typically has tertiary amino groups with pKa˜6.4. In plasma (pH 7.2), ˜10% are cationic, whereas inside endosomes (pH 5), >90% are cationic, which drives escape. In certain embodiments, a cationic and/or ionizable lipid is selected from the group consisting of KL22, KL25, 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA or MC3), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3)-cholest-5-en-3-yloxy]octyl}oxy)-N,N-dimethyl-3-[(9Z,12Z)-octadeca-9, 12-dien-1-yloxy]prop an-1-amine (Octyl-CLinDMA (2S)).

As used herein, the term “linker” or “coupler” refers to a domain of a larger molecule that connects one portion of the larger molecule to another portion of the larger molecule. In one embodiment, the linker is biocompatible and/or biodegradable. In another embodiment, the biodegradable linker comprises a thioether group, a carbamate group, an ester group, a carbonate group, a urea group, or an enzyme cleavable peptidic linkage. In yet another embodiment, the biodegradable linker is an endosomal cleavable linker. In a further embodiment, the endosomal cleavable linker comprises a carbamate group or a hydrazone group. In yet a further embodiment, the linker comprises a group selected from an optionally substituted (C₁-C₆) alkyl, an optionally substituted (C₂-C₆) alkenyl, an optionally substituted (C₂-C₆) alkynyl, or an optionally substituted (C₁-C₆) alkoxy group. In another embodiment, the linker comprises a group selected from ethyl, propyl, PEG₂, PEG₃ and PEG₄. In regards to the endosomal cleavable or degradable linker, the linker is susceptible to action of enzymes, or environments found in a subject's body. Such enzymes include, but are not limited to, esterases, glycosidases, and peptidases. Environments found in the subject body can be reducing environments found in lysosomes. Examples of degradable linkers include, but are not limited to, a thioether group, a carbamate group, an ester group, a carbonate group, a urea group, or an enzyme cleavable peptidic linkage. In a particular embodiment, the biodegradable linker is an endosomal cleavable linker. An endosomal cleavable linker can be a self-immolating carbamate released as CO₂ or hydrozone or another endosome specific linker design. In one embodiment, a linker comprises a node leading to at least one dendrimer generation, wherein the dendrimer terminates at a hydrophilic domain or masked hydrophilic domain.

The term “non-release controlling excipient” as used herein, refers to an excipient whose primary function do not include modifying the duration or place of release of the active substance from a dosage form as compared with a conventional immediate release dosage form.

The term “optionally substituted” refers to a functional group, typically a hydrocarbon or heterocycle, where one or more hydrogen atoms may be replaced with a substituent. Accordingly, “optionally substituted” refers to a functional group that is substituted, in that one or more hydrogen atoms are replaced with a substituent, or unsubstituted, in that the hydrogen atoms are not replaced with a substituent. For example, an optionally substituted hydrocarbon group refers to an unsubstituted hydrocarbon group or a substituted hydrocarbon group.

The term “peptide,” as used herein, represents two to about 50 amino acid residues linked by peptide bonds. The term “polypeptide,” as used herein, represents chains of 50 or more amino acids linked by peptide bonds. Moreover, for purposes of this disclosure, the term “polypeptide” and the term “protein” are used interchangeably herein in all contexts, unless provided for otherwise, e.g., naturally occurring or engineered proteins. A variety of polypeptides may be used within the scope of the methods and compositions provided herein. In a certain embodiment, polypeptides include antibodies or fragments of antibodies containing an antigen-binding site. In other embodiments, a polypeptide can include enzymatically active entities (e.g., Cas protein) and the like. Polypeptides made synthetically may include substitutions of amino acids not naturally encoded by DNA (e.g., non-naturally occurring or unnatural amino acid). Examples of non-naturally occurring amino acids include D-amino acids, an amino acid having an acetylaminomethyl group attached to a sulfur atom of a cysteine, a pegylated amino acid, the omega amino acids of the formula NH₂(CH₂)ₘCOOH wherein n is 2-6, neutral nonpolar amino acids, such as sarcosine, t-butyl alanine, t-butyl glycine, N-methyl isoleucine, and norleucine.

The term “pharmaceutically acceptable carrier,” “pharmaceutically acceptable excipient,” “physiologically acceptable carrier,” or “physiologically acceptable excipient” as used herein, refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. Examples of “pharmaceutically acceptable carriers” and “pharmaceutically acceptable excipients” can be found in the following. Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004.

The term “polynucleotide” or “nucleic acid” and “oligonucleotide” as used herein, represents two or more nucleotides, nucleosides or synthetic nucleotide derivatives covalently bound together by an internucleotide or bridging group. Polynucleotides may be linear or circular. Moreover, for the purposes of this disclosure, the term “polynucleotide” is in reference to both oligonucleotides and longer sequences, and to mixtures of nucleotides, e.g., mixtures of DNA and RNA or mixtures of RNA and 2′ modified RNA or Phosphorodiamidate Morpholino Oligomer (PMO) and Peptide Nucleic Acid (PNA), other nucleic acid derivatized oligonucleotides and polymers. The term “polynucleotide” encompasses polynucleotides which are comprised of one or more strands, unless stated otherwise. The term polynucleotide includes DNA, RNA, PMO, PNA, other nucleic acid derivatized oligonucleotides and polymers, including double stranded and single stranded forms thereof, hybrids and the like of any combination of these oligonucleotides.

The term “protecting group.” as used herein, represents a group intended to protect a functional group (e.g., a hydroxyl, an amino, or a carbonyl) from participating in one or more undesirable reactions during chemical synthesis (e.g., polynucleotide synthesis). The term “O-protecting group,” as used herein, represents a group intended to protect an oxygen containing (e.g., phenol, hydroxyl or carbonyl) group from participating in one or more undesirable reactions during chemical synthesis. The term “N-protecting group,” as used herein, represents a group intended to protect a nitrogen containing (e.g., an amino or hydrazine) group from participating in one or more undesirable reactions during chemical synthesis. Commonly used O- and N-protecting groups are disclosed in Greene, “Protective Groups in Organic Synthesis,” 3^{rd} Edition (John Wiley & Sons, New York, 1999), which is incorporated herein by reference. Exemplary O- and N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl. The use of protecting groups can be used to make the hydrophilic-masked cationic charge dendrimers and LNPs of the disclosure.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and alkaryl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxy benzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxy benzyl; 3,4-dimethoxy benzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methox benzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, alkaryl groups such as benzyl, triphenylmethyl, benzyloxymethyl, and the like and silyl groups such as trimethylsilyl, and the like. Useful N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “release controlling excipient” as used herein, refers to an excipient whose primary function is to modify the duration or place of release of the active substance from a dosage form as compared with a conventional immediate release dosage form.

The term “subject” as used herein, refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein. For example, a mammalian subject can refer to a human subject or patient.

The term “substituent” refers to an atom or group of atoms substituted in place of a hydrogen atom. For purposes of this invention, a substituent would include deuterium atoms.

The term “substituted” with respect to hydrocarbons, heterocycles, and the like, refers to structures wherein the parent chain contains one or more substituents.

The term “targeting moiety,” as used herein, represents any moiety that specifically binds or reactively associates or complexes with a receptor or other receptive moiety associated with a given target cell population or a moiety that induces endocytosis when contact with a cell or is endocytosed by a cell.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenicity, are commensurate with a reasonable benefit/risk ratio, and are effective for their intended use.

The terms “treat”, “treating” and “treatment”, as used herein, refers to ameliorating symptoms associated with a disease or disorder (e.g., cancer), including preventing or delaying the onset of the disease or disorder symptoms, and/or lessening the severity or frequency of symptoms of the disease or disorder.

The term “unsubstituted” with respect to hydrocarbons, heterocycles, and the like, refers to structures wherein the parent chain contains no substituents.

The ability to deliver functional agents to cells is problematical due to the bioavailability restriction imposed by the cell membrane. That is, the plasma lipid bilayer membrane of the cell forms an effective barrier, which restricts the intracellular uptake of molecules to those which are sufficiently non-polar and smaller than approximately 500 Daltons in size. Previous efforts to enhance the internalization of proteins have focused on fusing proteins with receptor ligands (Ng et al., Proc. Natl. Acad. Sci. USA, 99:10706-11, 2002) or by packaging them into caged liposomal carriers (Abu-Amer et al., J. Biol. Chem. 276:30499-503, 2001). However, these techniques often result in poor cellular uptake and intracellular sequestration into the endocytic pathway. In addition, liposomal formulations can be cytotoxic.

All intracellular macromolecular therapeutics (including: siRNAs, ASOs, PMOs, PNAs, Peptides, Proteins, Large Synthetic Molecules, CRISPR, RNPs, mRNA, RNAs, DNA vector, LNPs, NPs, etc.) are taken up by cells by various forms of endocytosis. Endosomes comprise a lipid bilayer membrane barrier that prevents >95% or >99% or more of macromolecular therapeutic from escaping the endosome and entering the cytoplasm and nucleus of cells. Thus, endosomal escape remains a significant technological problem that needs to be solved to enable deliver of all macromolecular therapeutics. Enveloped viruses also have to address the endosomal escape problem and use a protein machine that contains an outer hydrophilic mask covering an inner hydrophobic endosomal escape domain.

PTDs/CPPs have been used to deliver therapeutic cargo into cells in culture, studied in pre-clinical models of disease and are currently in clinical trials. There are over 100 published PTD/CPP delivery domain sequences; however, most published PTDs/CPPs have been investigated using dye-labeled molecules. Consequently, excluding cell death, there is a paucity of quantitative transduction assays that rely on robust and well-controlled cellular phenotypes that can be readily quantified to determine which PTDs/CPPs are the most efficient and least cytotoxic delivery domains. Briefly, PTD/CPP delivery of macromolecules into the cytoplasm requires: (1) cellular association and uptake by endocytosis, and (2) escape from the endosome into the cytoplasm, which is the rate-limiting delivery step. It is estimated that only a small fraction of the endosomal bound (cell associated) TAT-PTD/CPP escapes from the macropinosome into the cytoplasm, perhaps as little as or even less than 1%.

The disclosure provides hydrophilic-masked cationic charge dendrimers that solve the endosomal escape problem by being acted on by endosomal/lysosomal enzymes to unmask cationic charge dendrimers that facilitate endosomal escape. Further, the approaches and methods disclosed herein can be applied directly to lipids to make hydrophilic-masked cationic charge LNPs. The hydrophilic-masked cationic charge dendrimers and LNPs of disclosure can be used to deliver a variety of macromolecules.

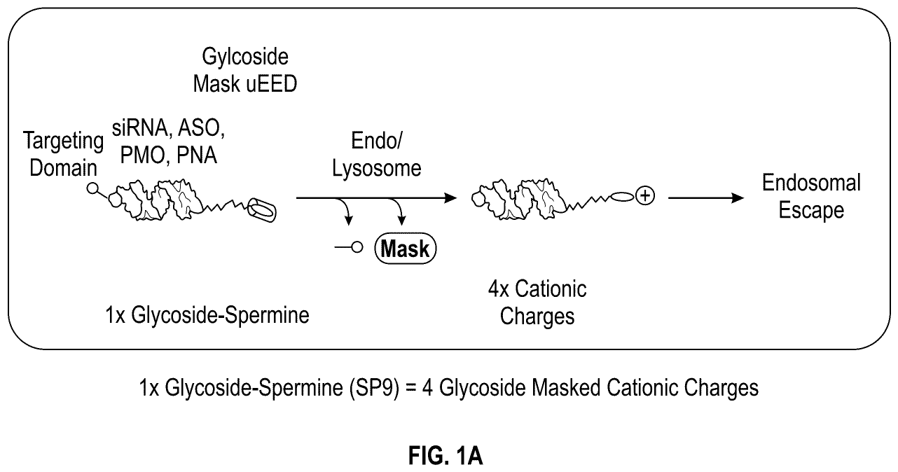

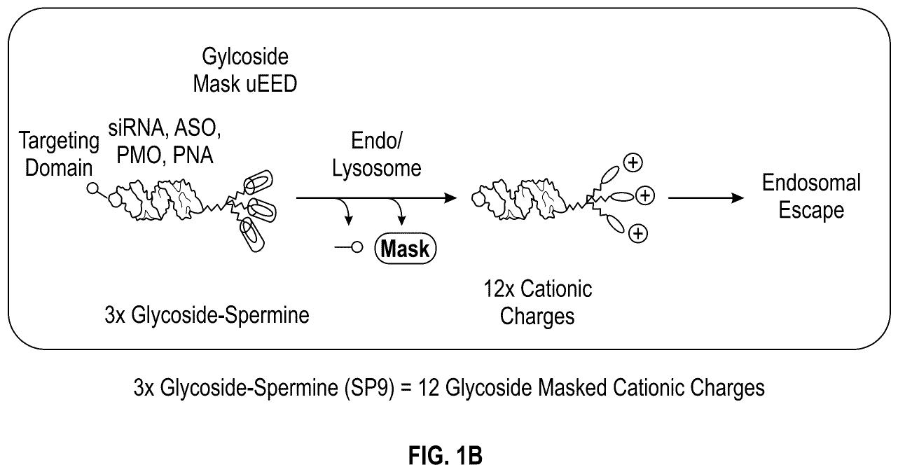

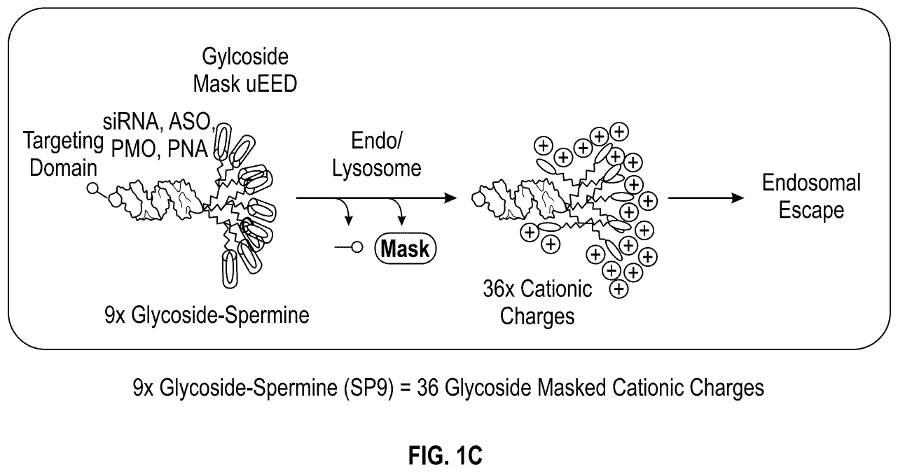

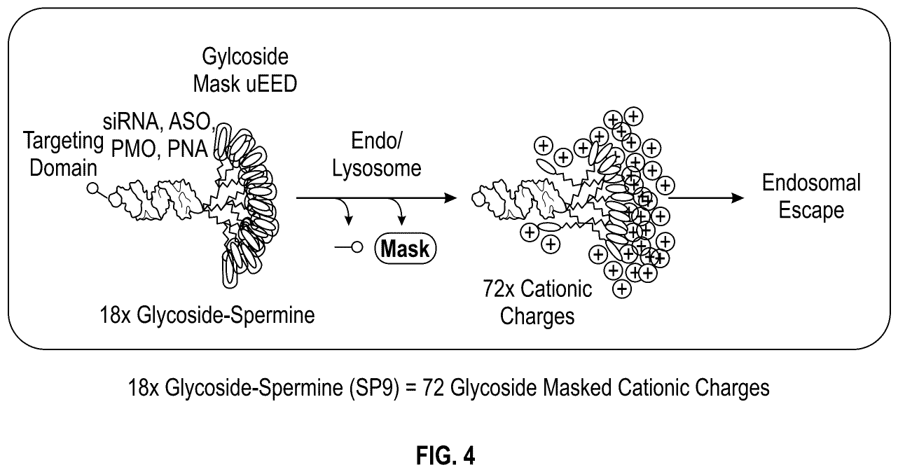

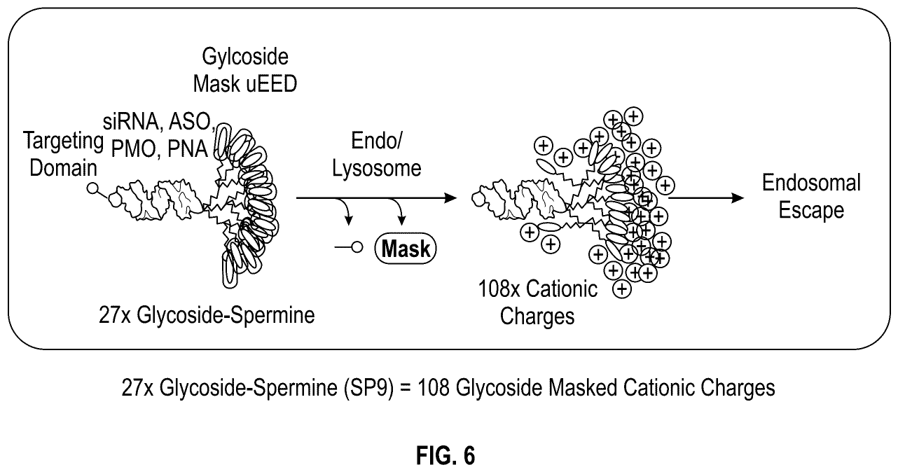

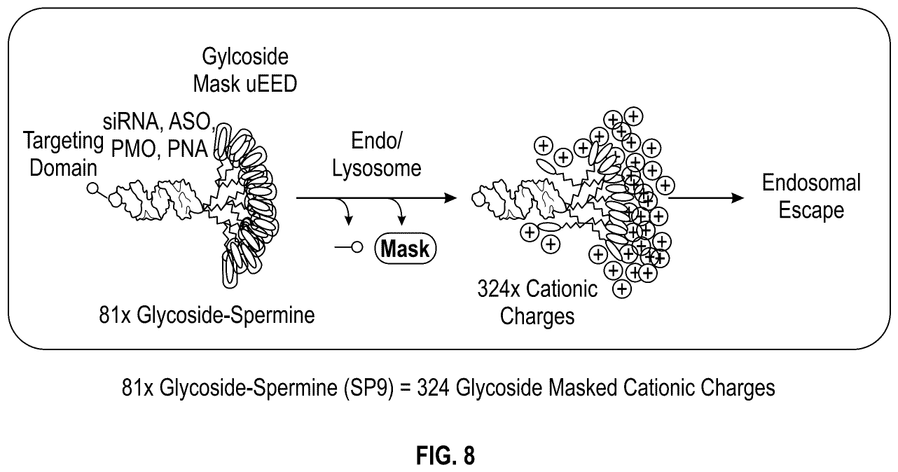

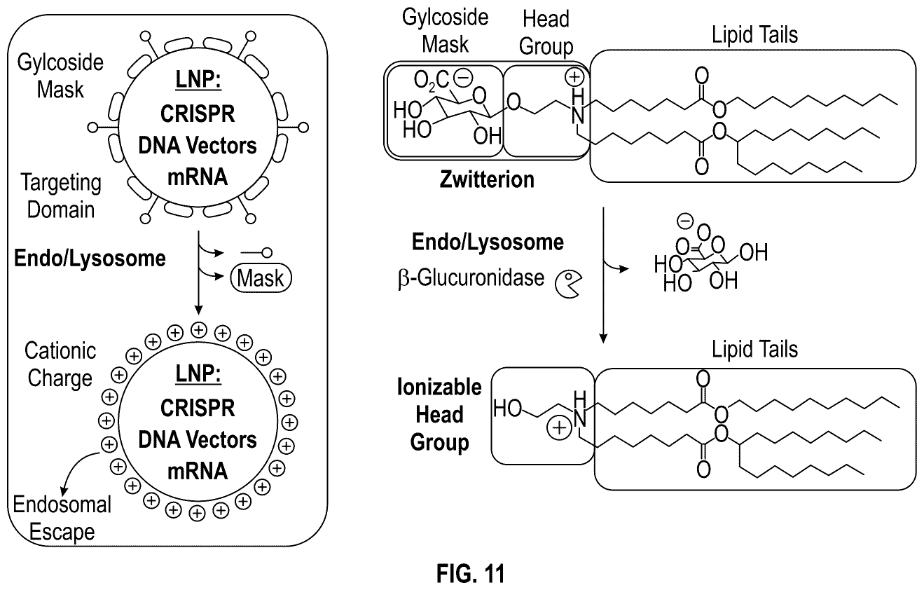

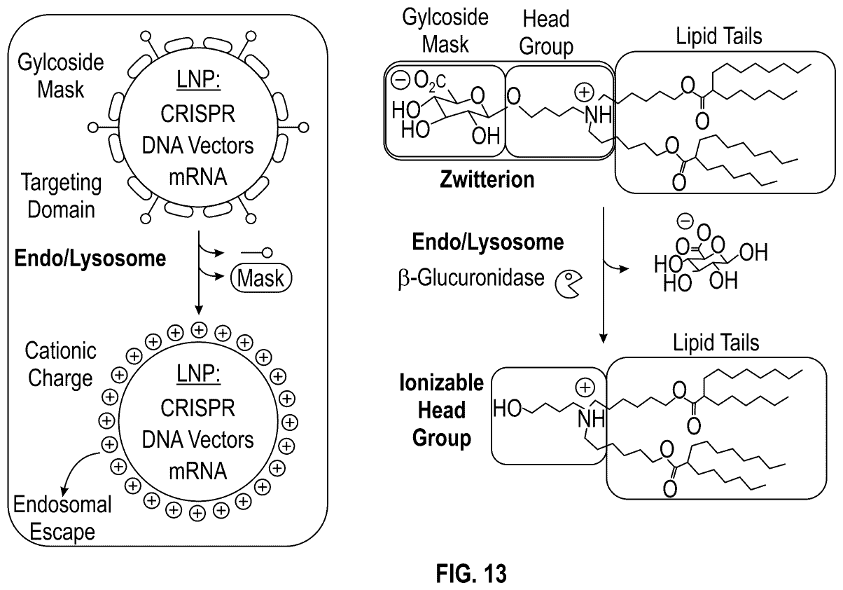

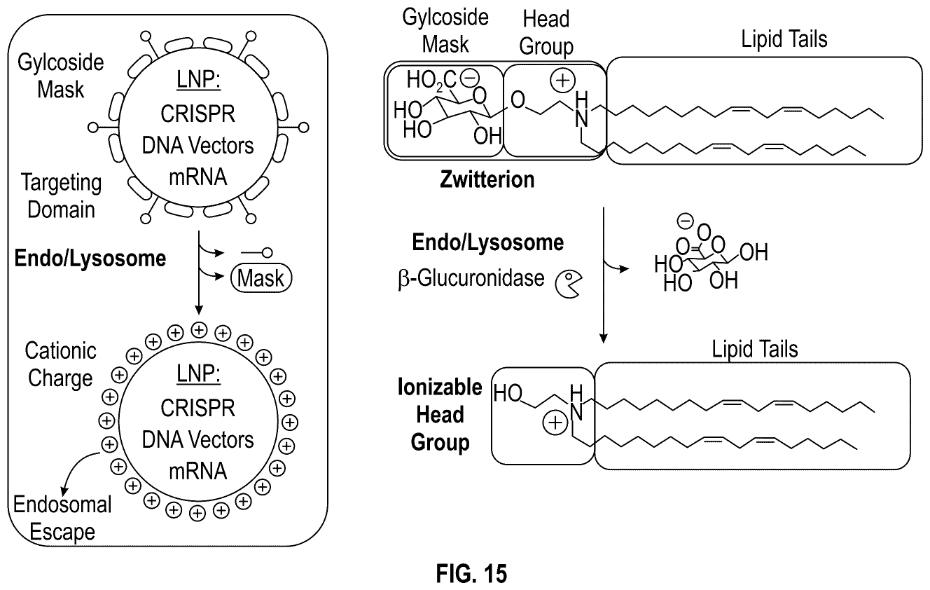

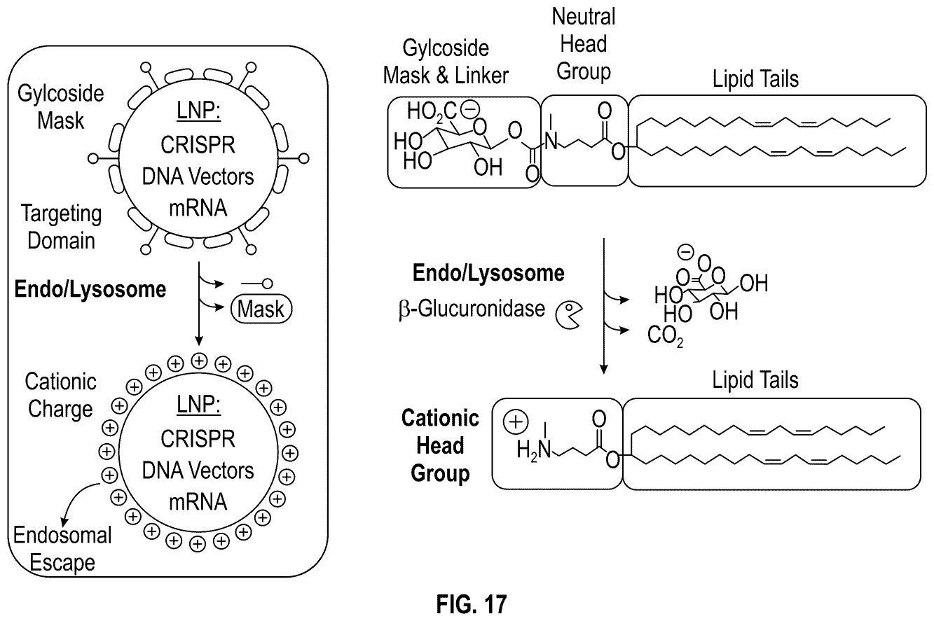

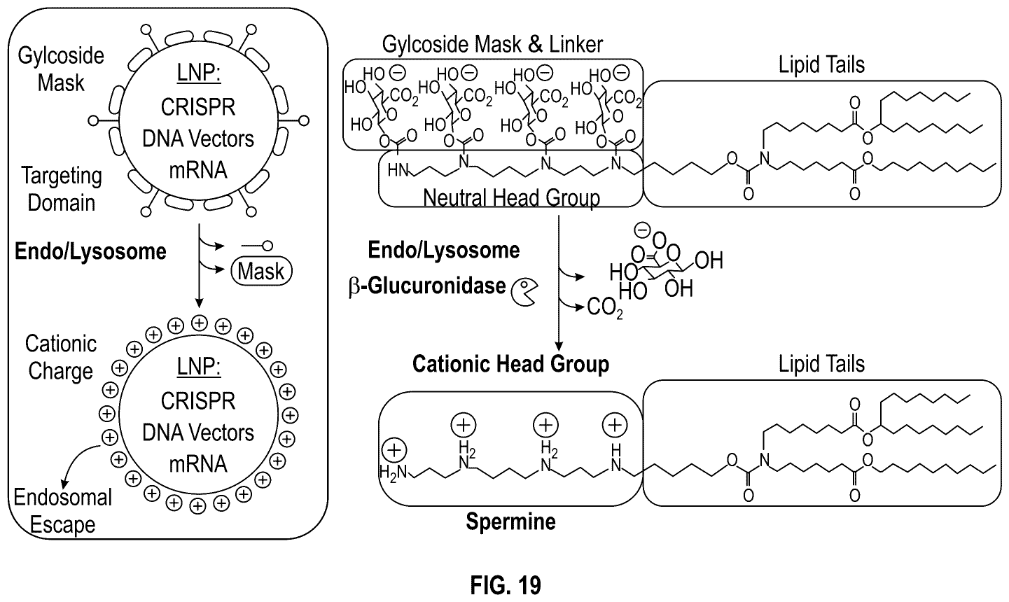

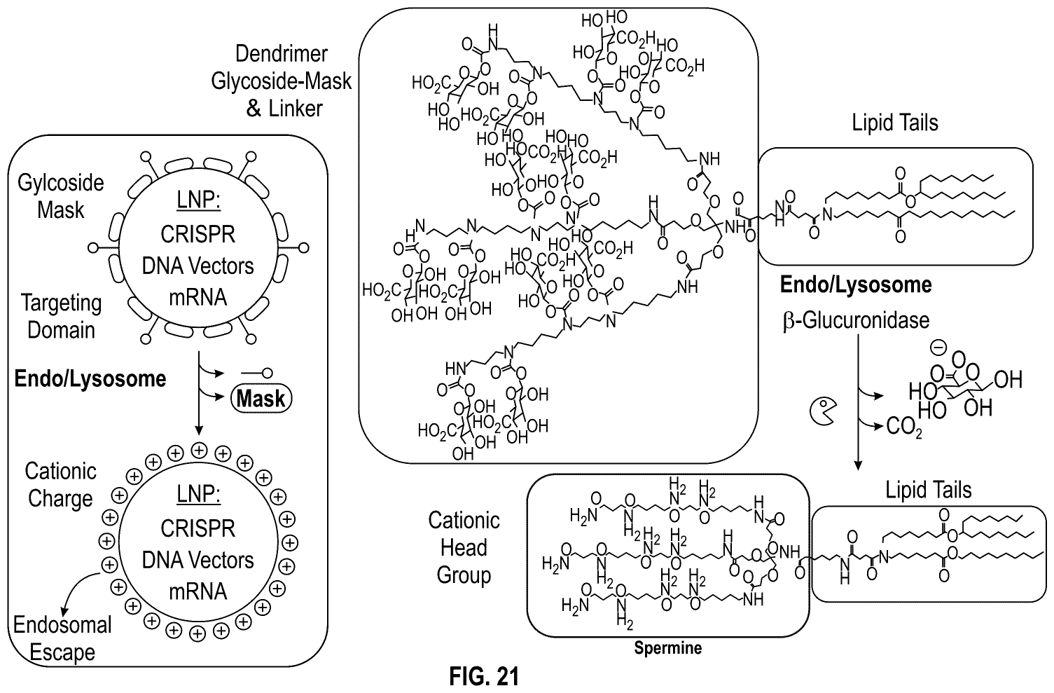

In a particular embodiment, the disclosure provides a composition comprising a hydrophilic-masked cationic charge dendrimer, or a hydrophilic-masked cationic charge LNP disclosed herein, which can be used to deliver cargo molecule(s). For example, the cationic charge dendrimer may be linked or complexed with cargo molecule(s) (e.g., see FIGS. 1, 4, 6, and 8); while the LNPs may be loaded with cargo molecule(s) (e.g., see FIGS. 11, 13, 15, 17, 19, and 21). Examples of cargo molecules, can include macromolecules, such as nucleic acids, oligonucleotides, vectors, gene editing systems, and therapeutics. The hydrophilic-masked cationic charge dendrimers can be acted on by lysosomal/endosomal enzymes to unmask cationic charged dendrimers that are linked to cargo(s). The hydrophilic-masked LNPs disclosed herein can be acted on by lysosomal/endosomal enzymes to unmask cationic charged head groups of the lipids. The unmasked cationic charged dendrimers or cationic charged lipids destabilize the membranes of the lysosome/endosome to promote escape of cargo molecules into the cytoplasm. The compositions of the disclosure therefore promote uptake and release a variety of macromolecules intracellularly.

The disclosure provides compositions useful in cellular transduction and cellular modulation. The cargo can be any number of different molecular entities including diagnostic and therapeutics for the treatment of a disease or disorder including small molecule and biologics for disease treatment. In one embodiment, the multi-domain approach can be used to deliver anticancer agents to a tumor cell and thereby kill tumor cells. The anti-cancer agent can be a peptide, polypeptide, protein, small molecule agent or an inhibitory nucleic acid (e.g., siRNA, ASO, PMO, PNA, oligonucleotide, ribozyme etc). In another embodiment, a macromolecular cargo can be delivered to a cell or tissue. Examples of macromolecular cargo including CRISPR/Cas systems, gRNA, adenosine deaminase acting on RNA (ADAR) and the like.

The disclosure provides compositions that comprise compounds that contain modular components that are operably linked such that each “component” or “domain” can serve a desired biological function. For example, a compound comprises a linker and/or coupler domain linked to a hydrophobic and/or cationic domain, wherein the hydrophobic and/or cationic domain is linked to a hydrophilic domain via a cleavable linker. Each module, e.g., the linker and/or coupler, the hydrophilic domain, the cleavable linker and the hydrophobic or cationic domain are functional for a particular purpose of releasing a cargo molecule intracellularly.

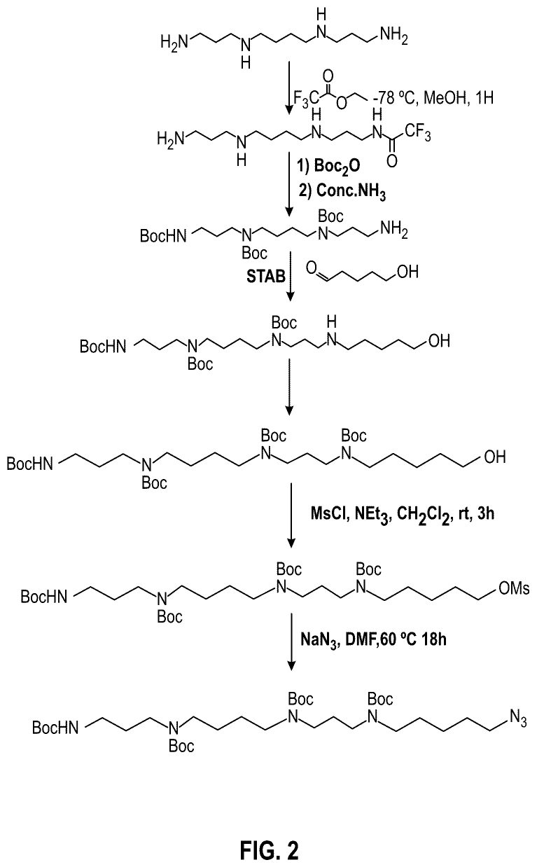

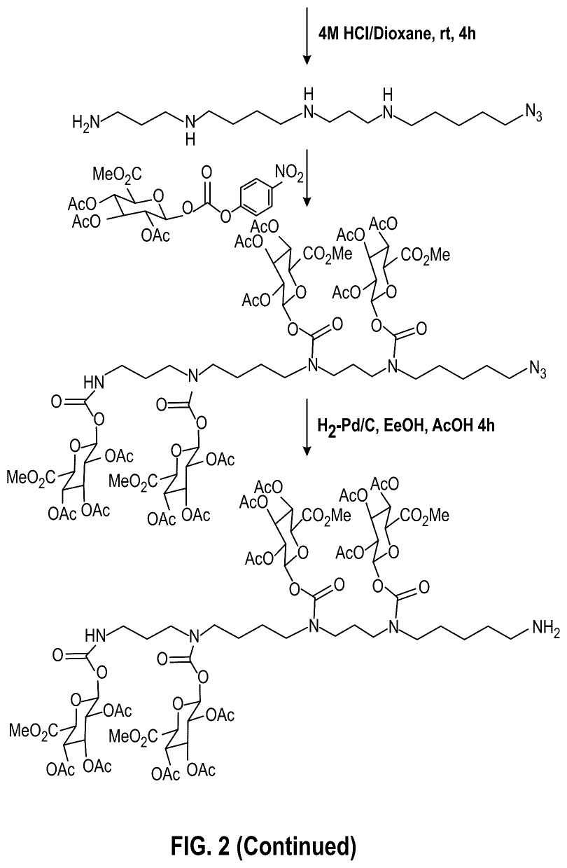

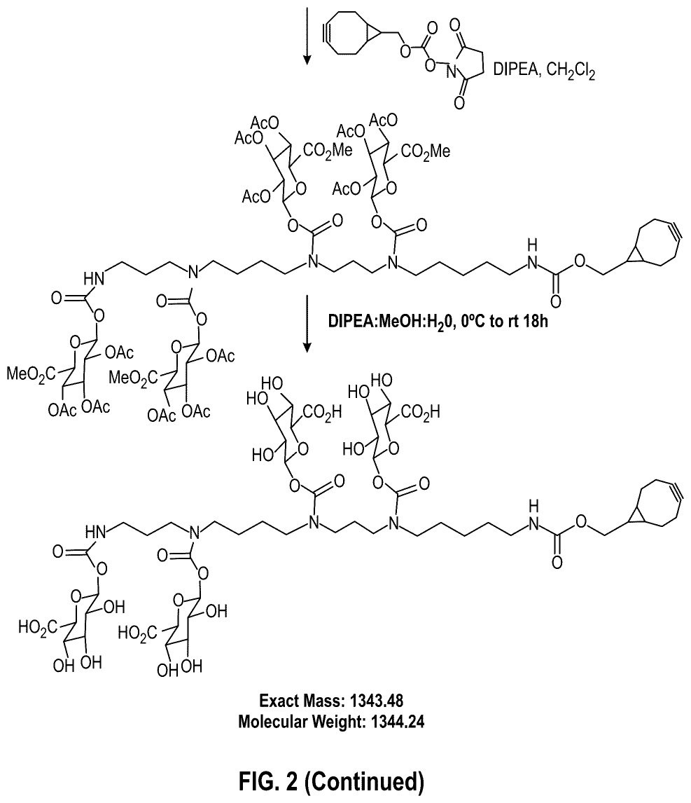

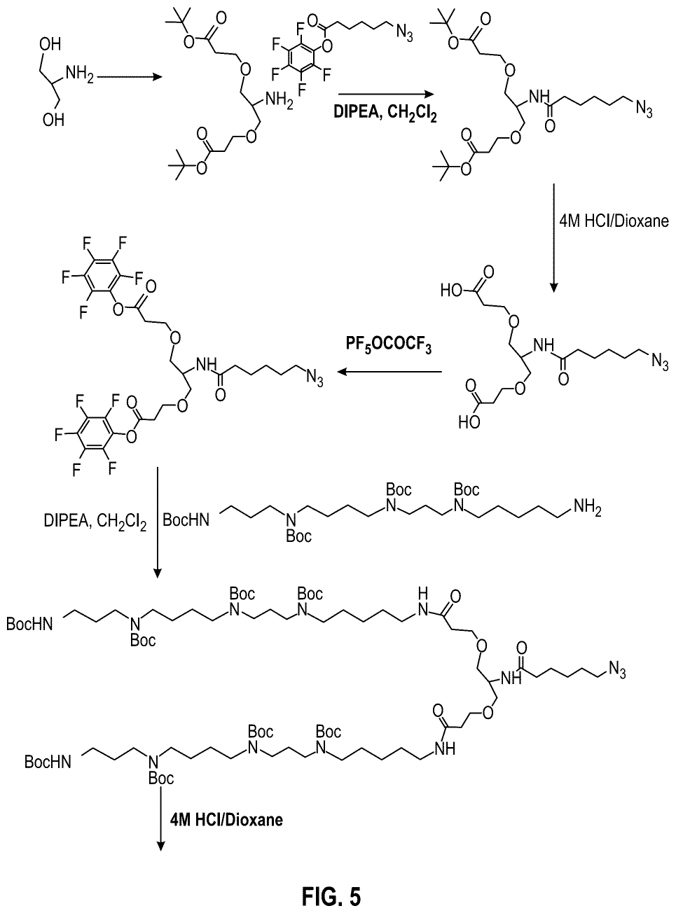

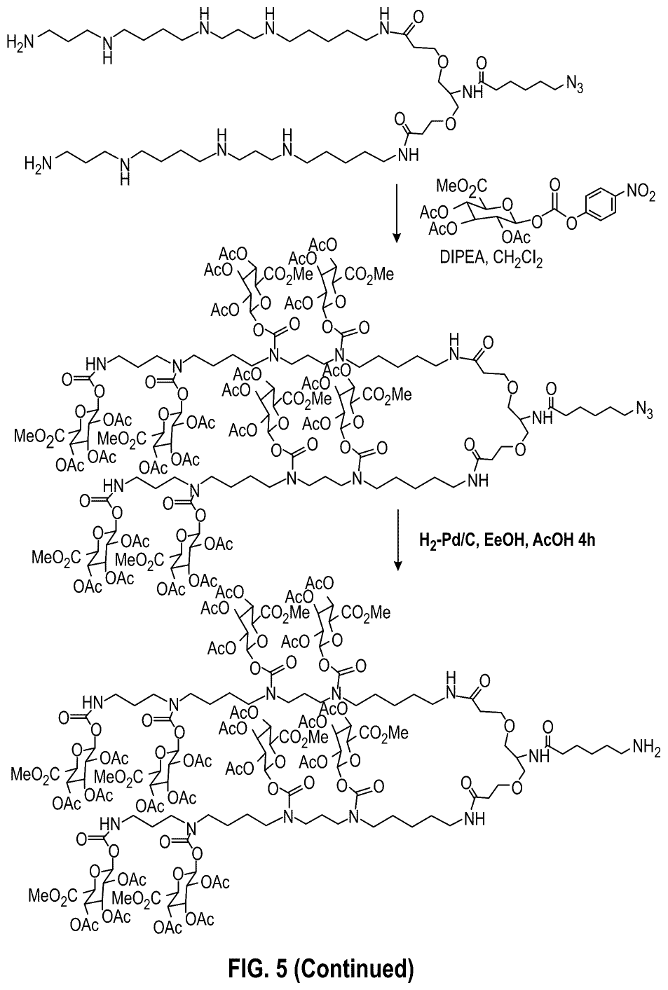

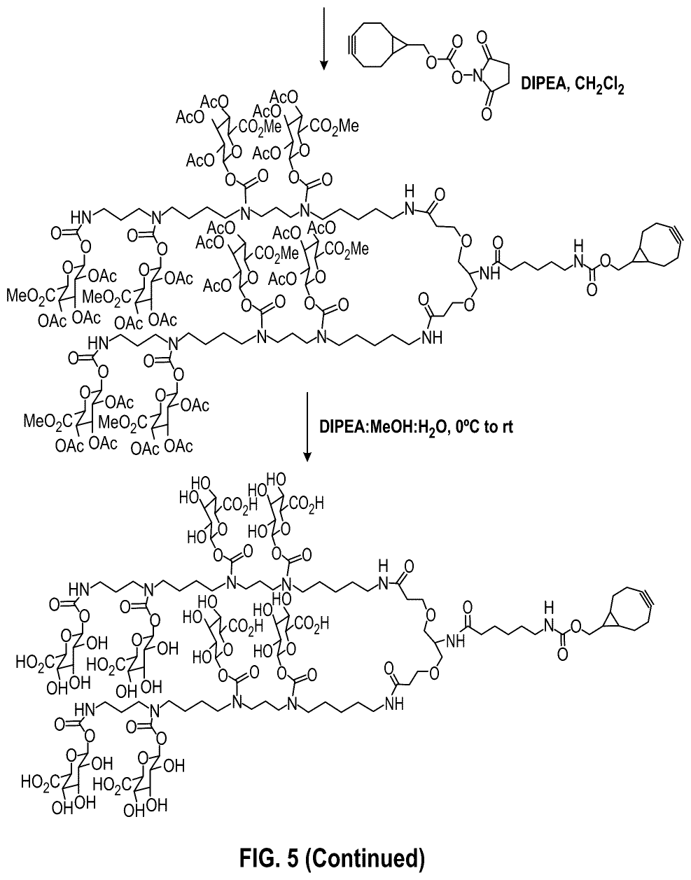

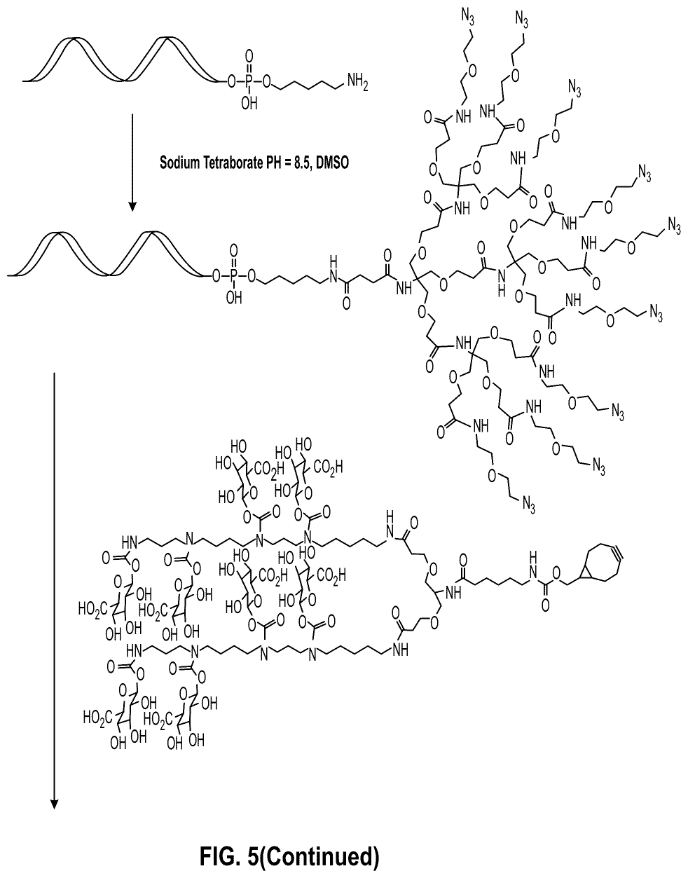

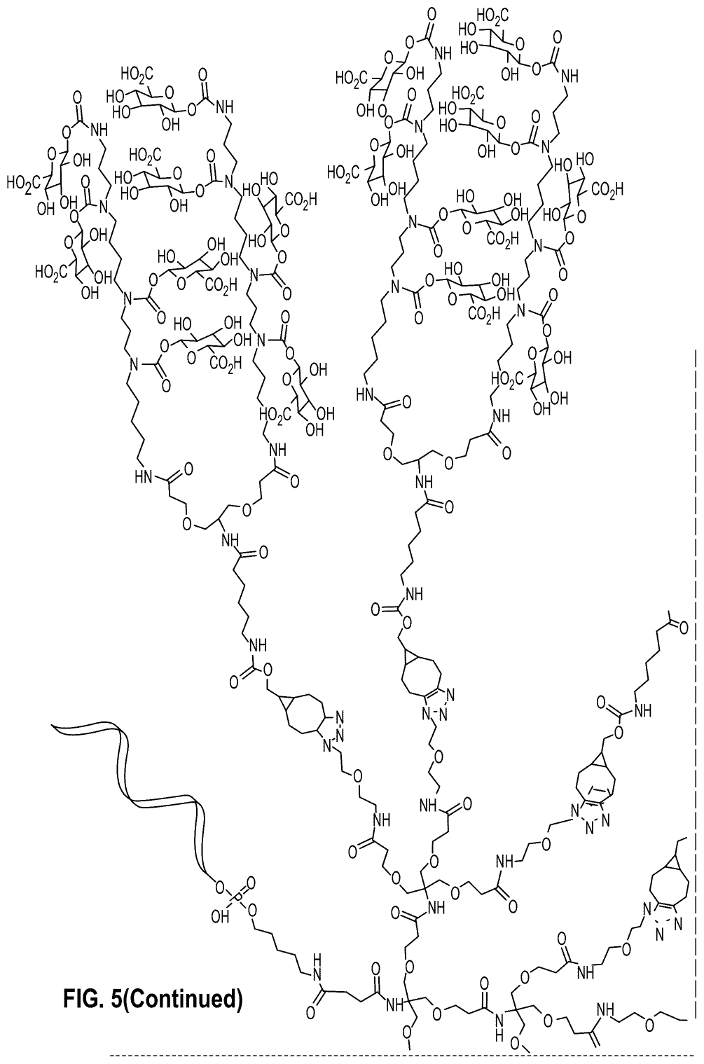

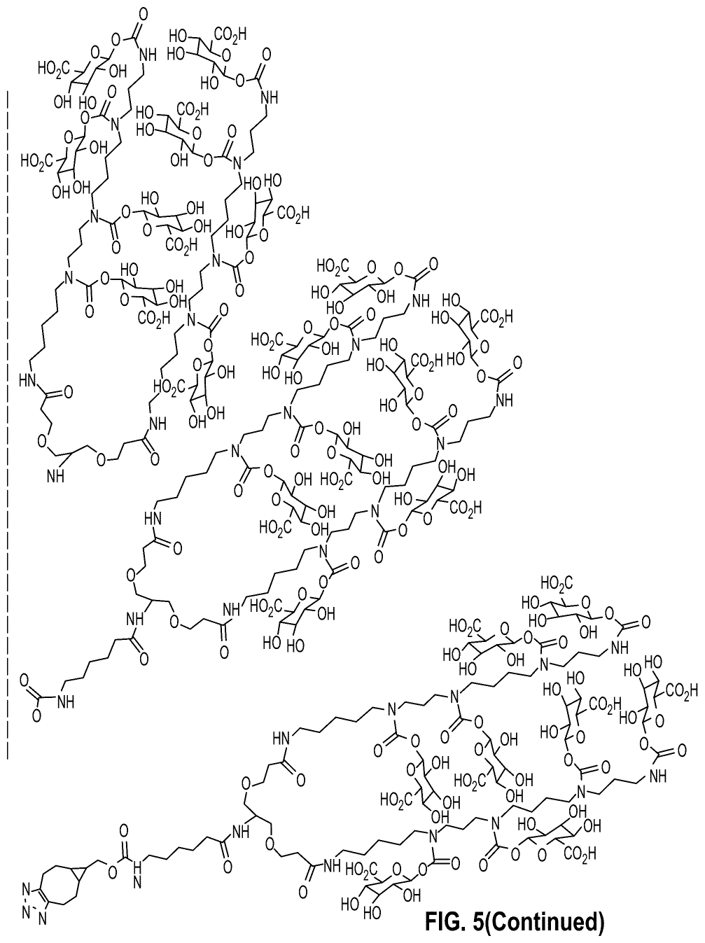

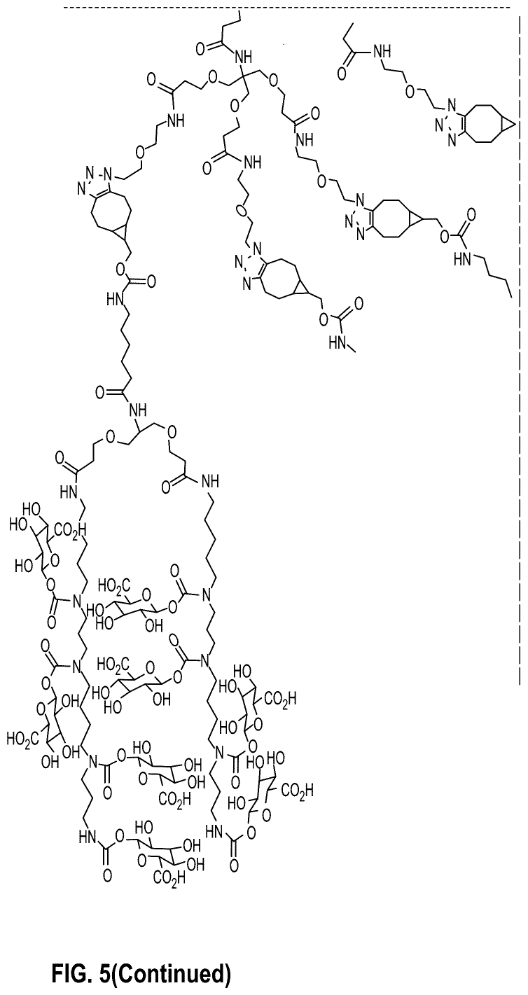

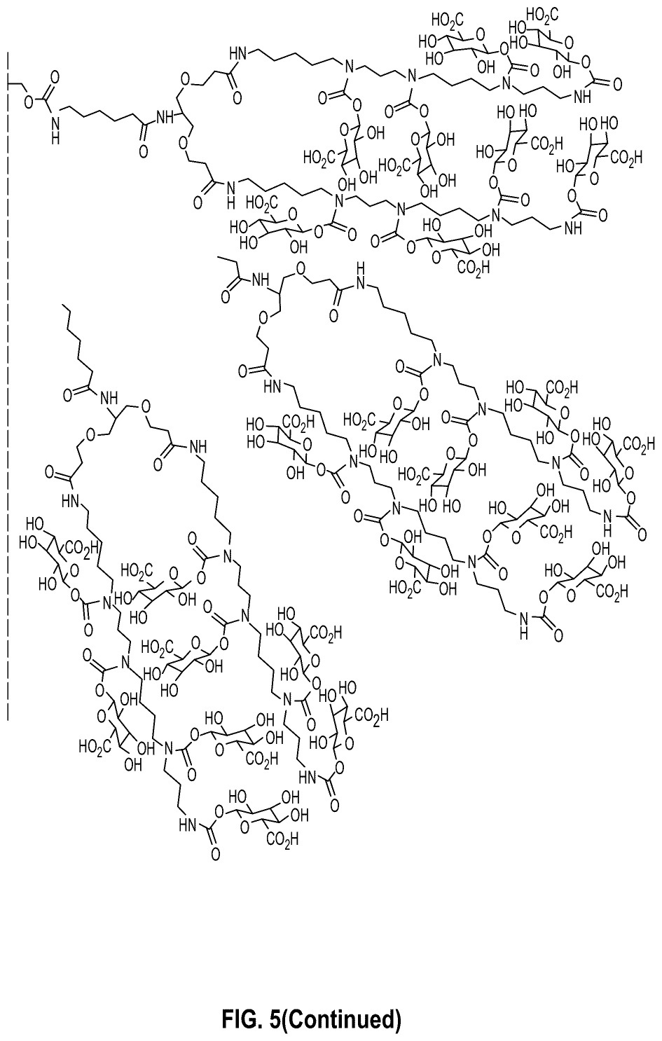

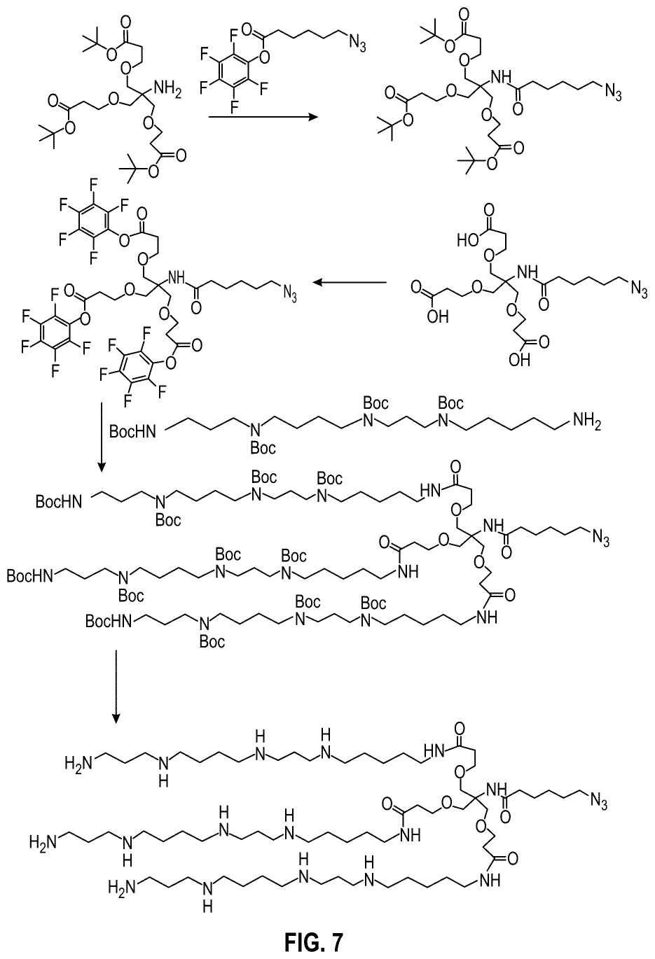

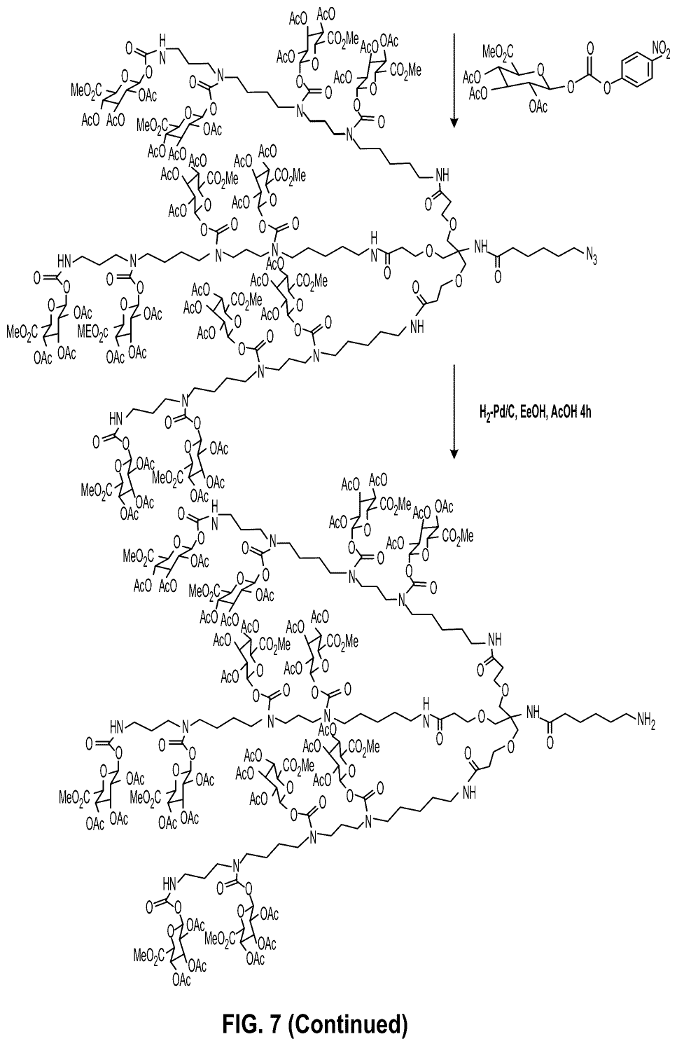

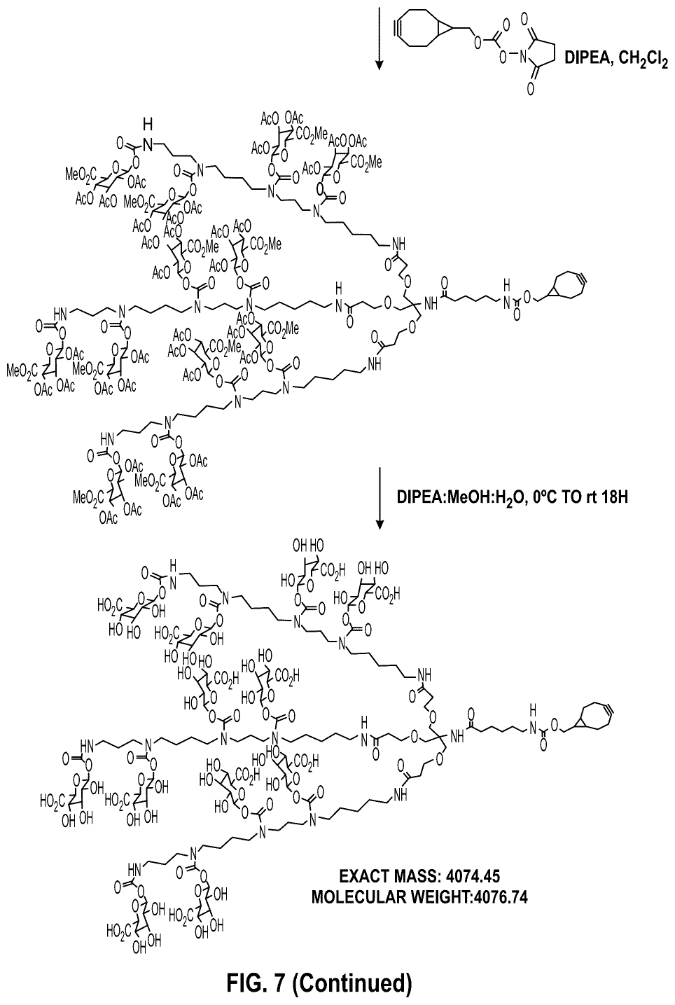

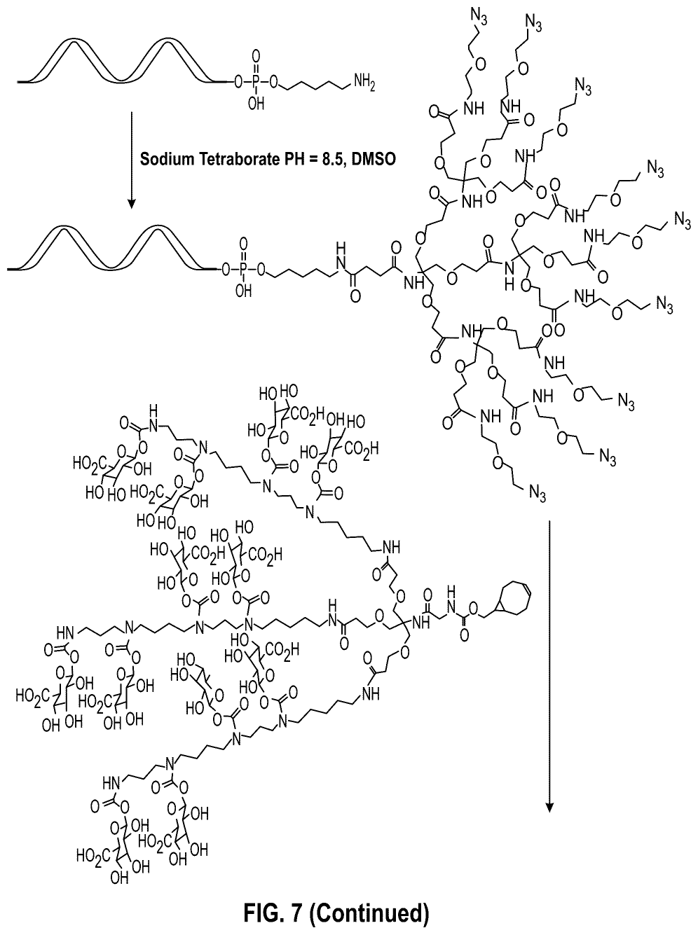

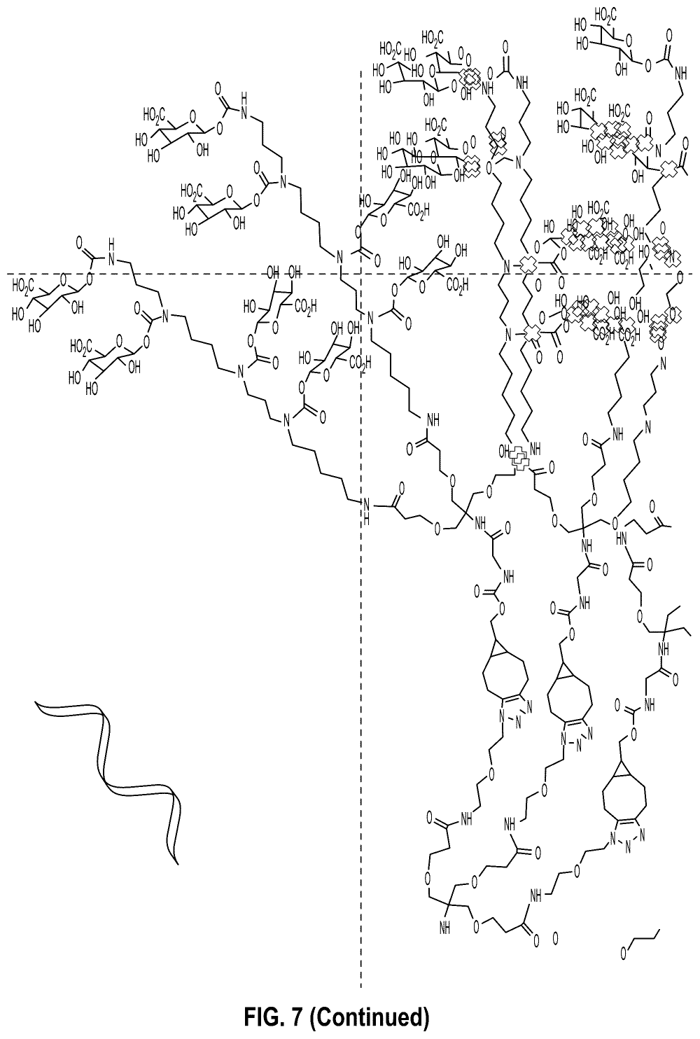

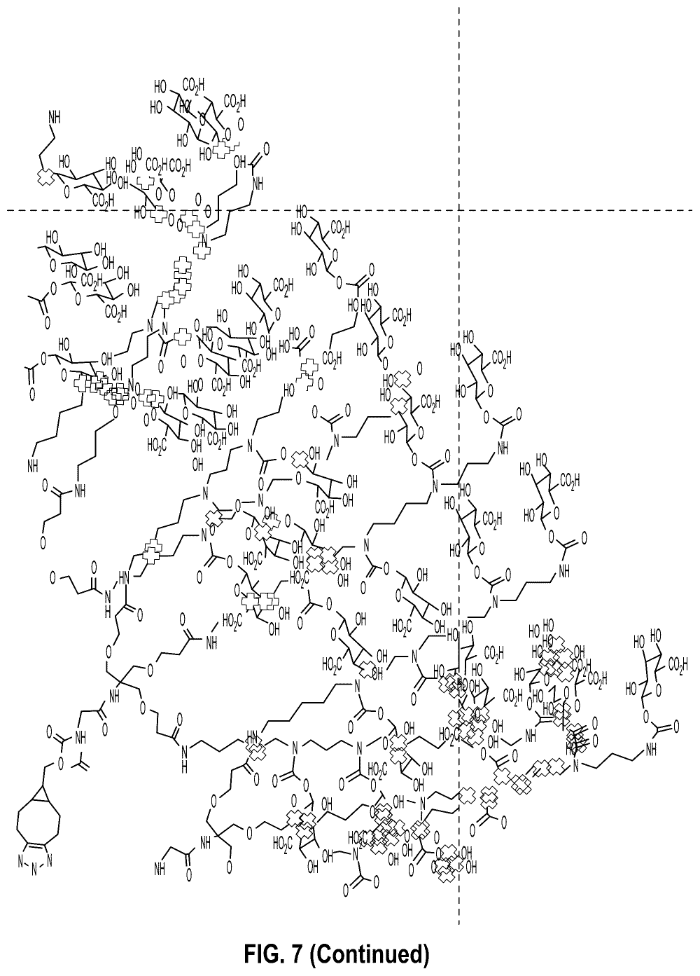

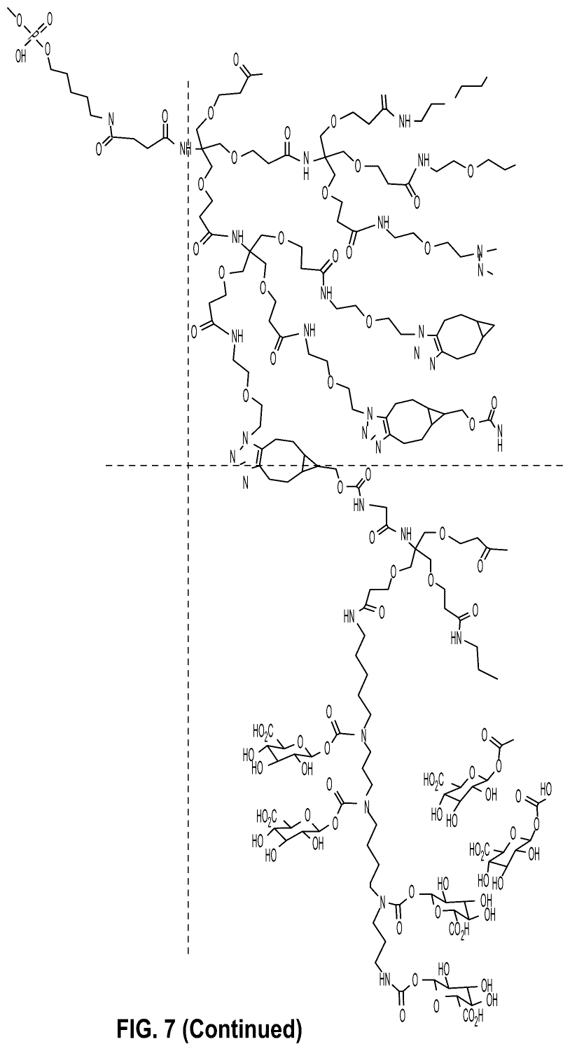

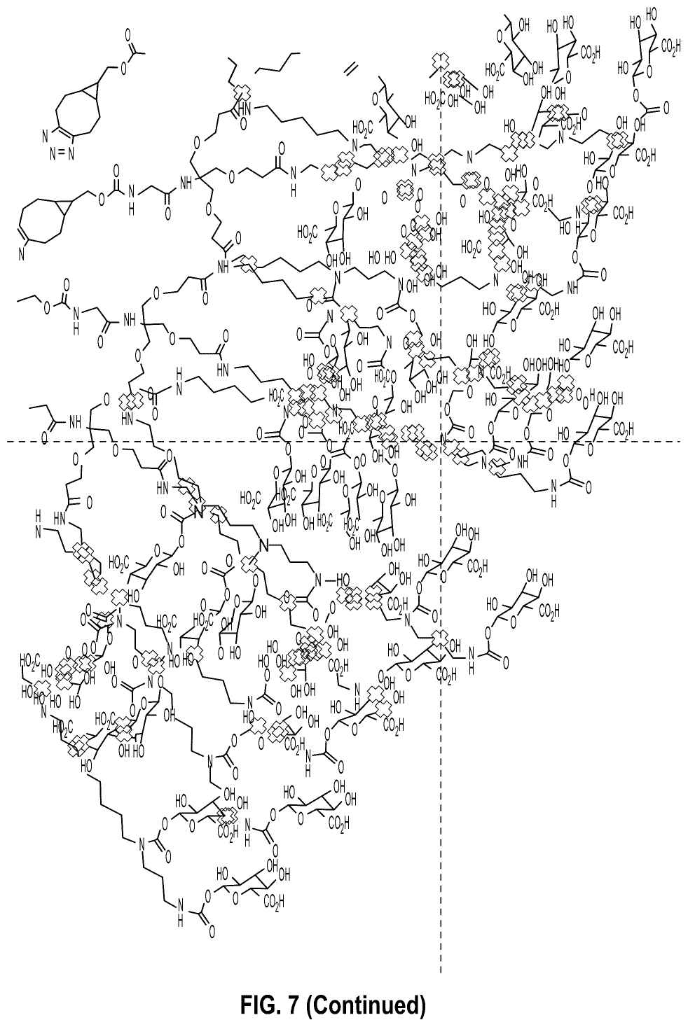

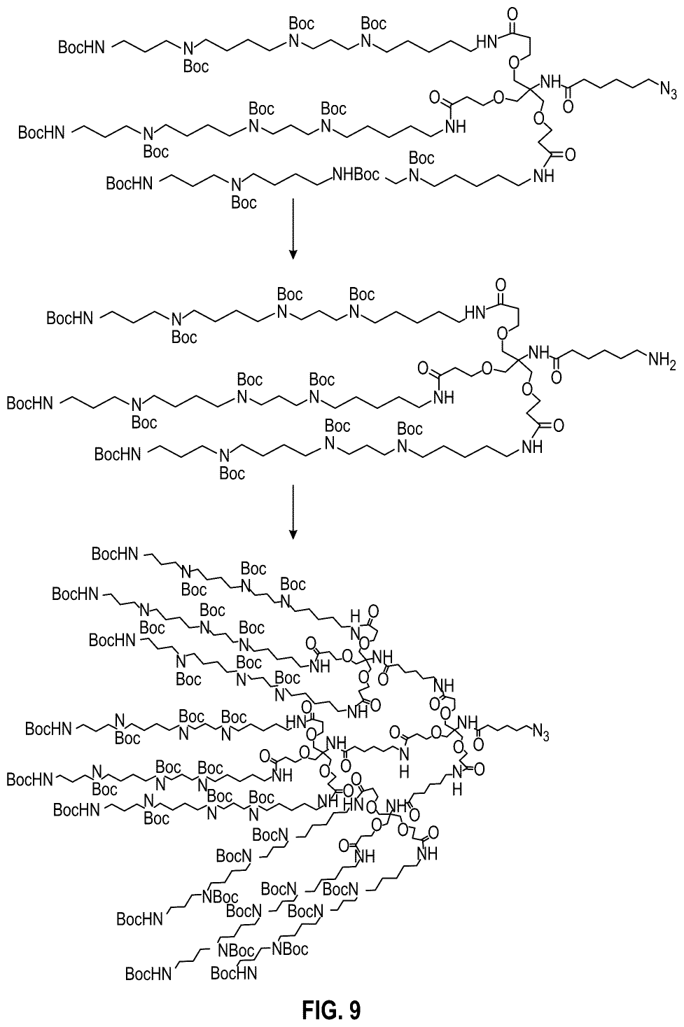

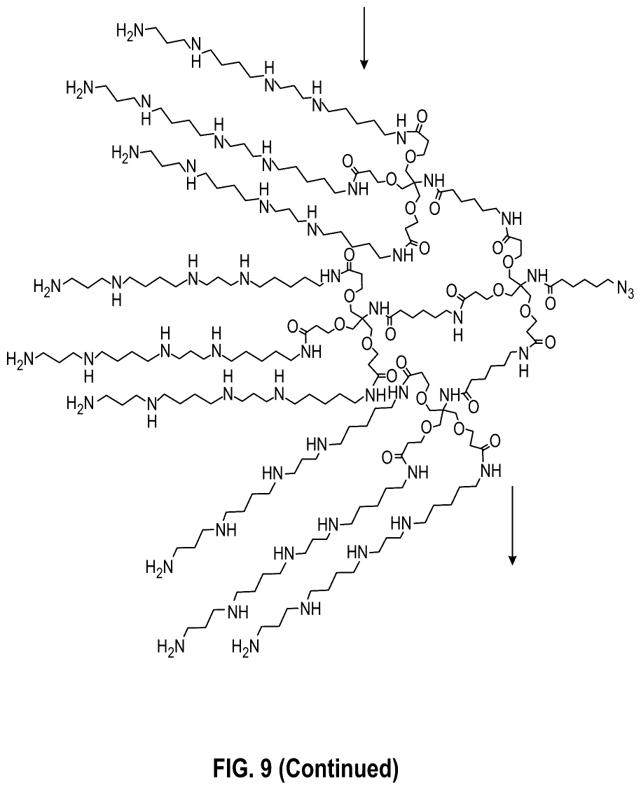

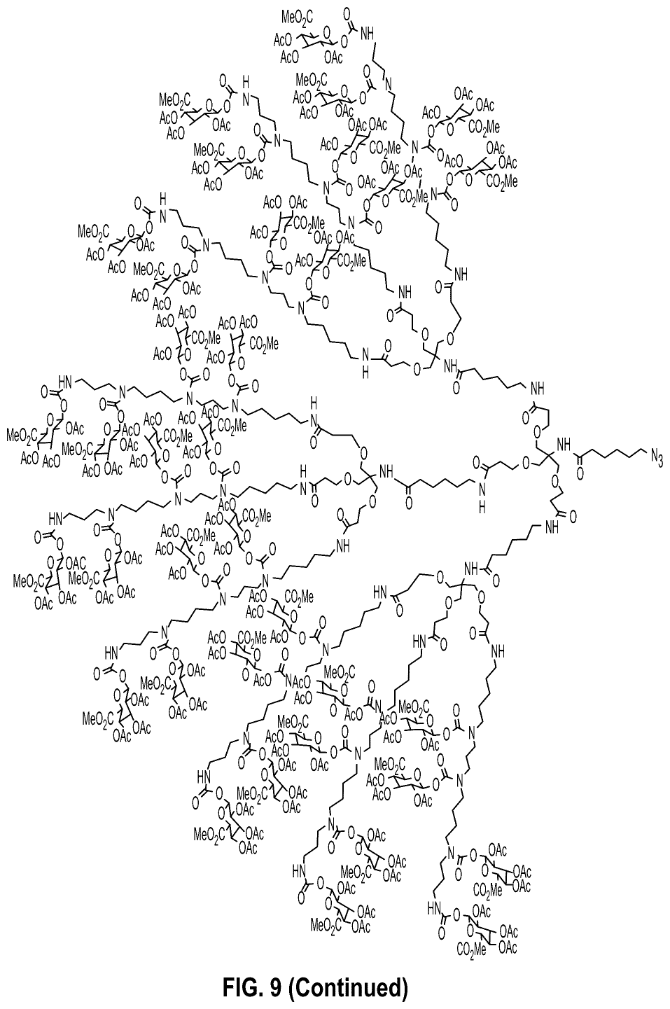

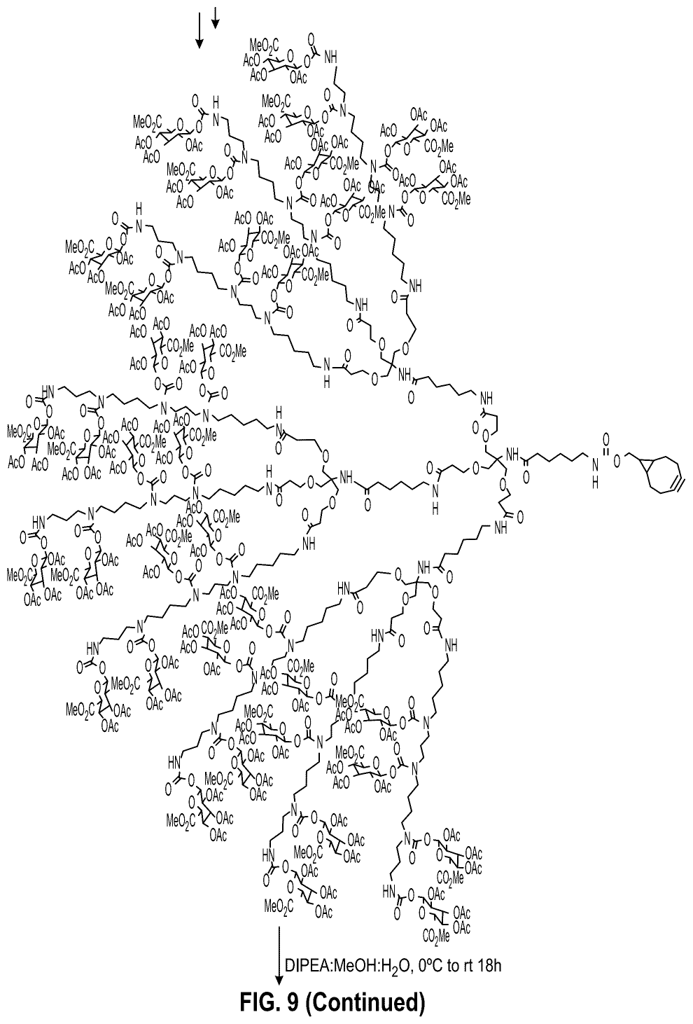

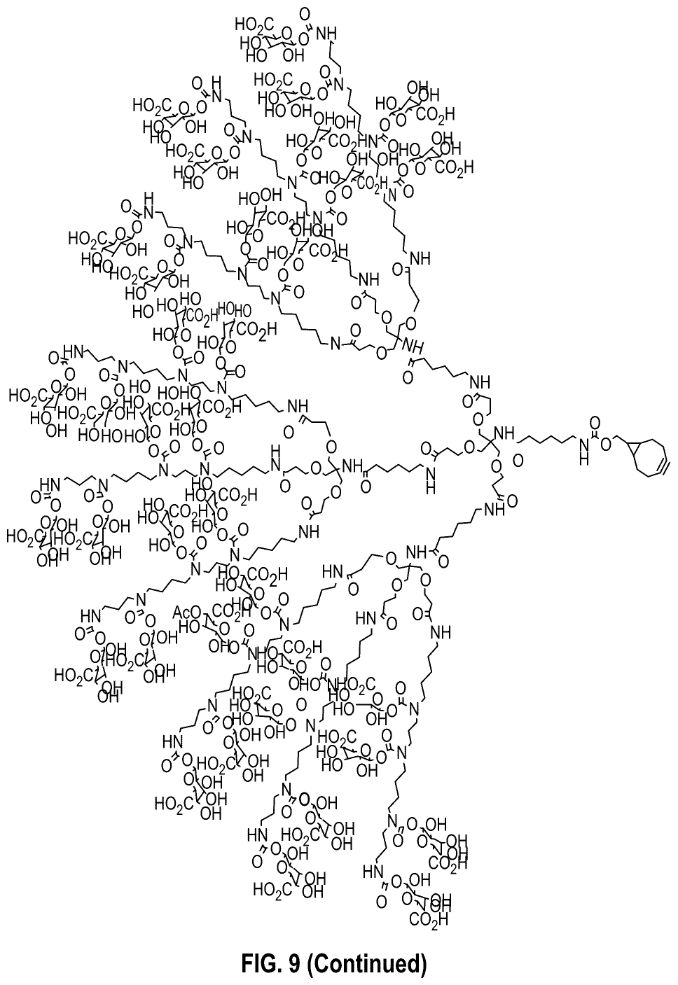

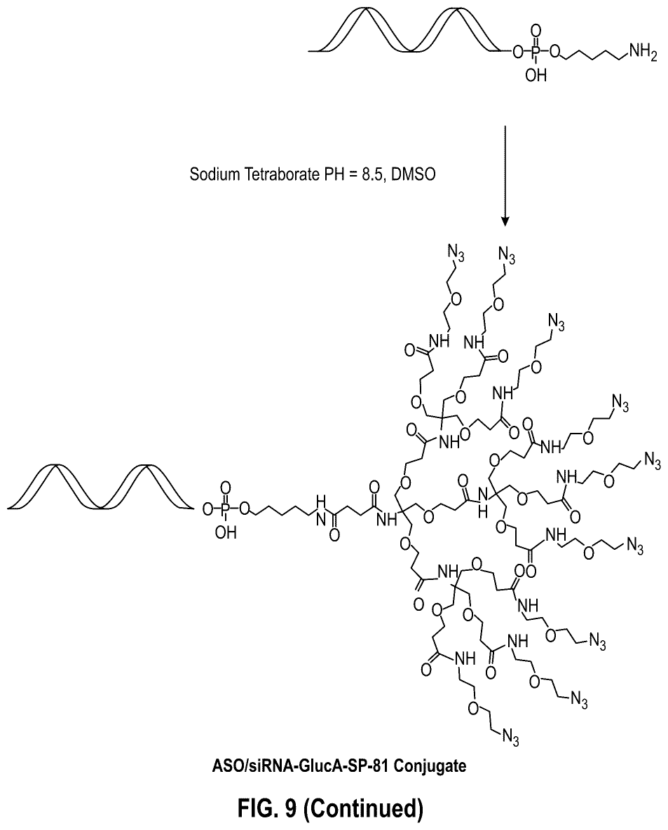

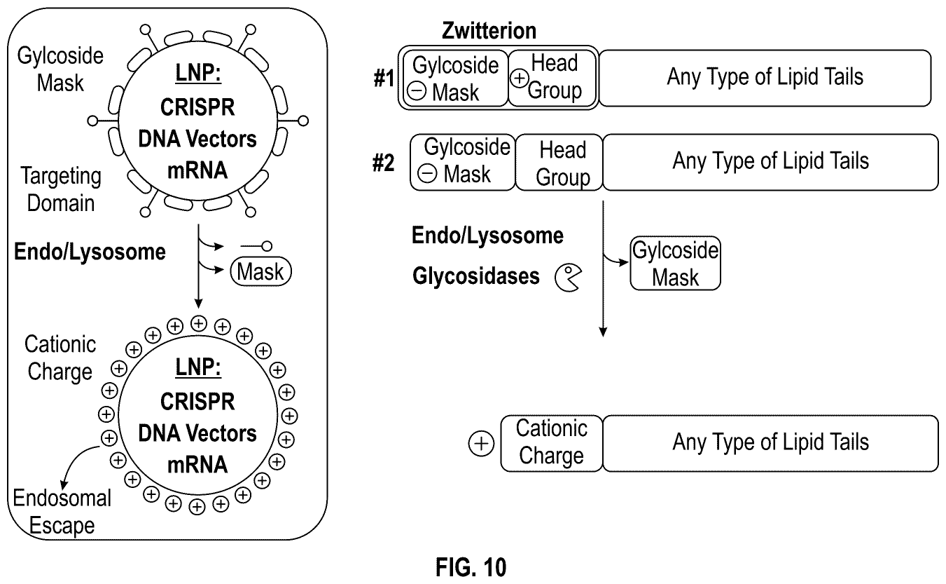

FIGS. 2, 3, 5, 7, and 9 provide exemplary hydrophilic-masked cationic charge dendrimers of the disclosure. As will be noted, the dendrimers can include different numbers of modular masking hydrophilic domains. An agent to be delivered (i.e., a cargo molecule) is linked to a portion of the dendrimers. A targeting moiety can be linked to the cargo moiety or the hydrophilic masking domain to promote endocytosis of the complex.

In regards to the hydrophilic masking group, this group is susceptible to enzymes and/or environments found in endosomes or lysosomes. For example, if the hydrophilic masking group comprises glycosides, the glycosides can be acted on by glycosidases found in endosomes or lysosomes. In regards to the hydrophilic mask domain of the compounds disclosed herein, this domain comprises a positive charge moiety, or becomes positively charged when the moiety is exposed to certain pH environments, e.g., physiological pH, or acidic environments. Examples of moieties that can be used for hydrophilic mask domains, include, but are not limited to, glycosides including, but not limited to, b-Glucuronic Acid, a/b Galactose, N-Acetyl Glucosamine, Sialic Acid, Xylose, N-Acetyl Galactosamine, Mannose, Glucose and other glycosides. The purpose of said moieties/domains is to mask the cationic charge dendrimer or lipid disclosed herein, and to increase solubility of the compounds in aqueous environments. As there are more than 40 types of glycosides that are specifically cleaved by endosomal restricted glycosidases, the use of glycoside for the hydrophilic mask domain provides for additional functionality.

In regards to the cationic charge dendrimers and cationic charge lipids, these molecules typically include one, typically a plurality of primary-secondary tertiary amino groups. All endosomes are composed of a lipid bilayer barrier that prevents >95% or >99% or more of macromolecular therapeutics from escaping the endosome and entering the cytoplasm and nucleus of cells. Thus, once the cationic charge dendrimer or cationic charge lipid is ‘unmasked’ by the removal of the hydrophilic mask domain inside the endosome/lysosome, the cationic charge dendrimer or cationic charge lipid will interact with or integrate into the endosomal membrane, thereby disrupting membrane integrity and promoting endosomal escape of linked or complexed cargo. In a particular embodiment, the cationic charge dendrimer and cationic charge lipid comprises moieties or headgroups that comprise one or more primary, secondary or tertiary amino groups. Other molecules including aromatic compounds such as indole and nitrogen containing aromatic multi-cyclic ring compounds are also contemplated.

The compounds or compositions of the disclosure provide for the delivery of cargo molecules that are operably linked or complexed with cationic charge dendrimers, or cargo molecules contained within hydrophilic masked cationic charge LNPs. The term “operably linked” refers to functional linkage between two domains (e.g., a dendrimer and a cargo molecule).

The cargo molecule can comprise a therapeutic agent and/or a diagnostic agent. Examples of therapeutic agents include, for example, thrombolytic agents and anticellular agents that kill or suppress the growth or cell division of disease-associated cells (e.g., cells comprising a cell proliferative disorder such as a neoplasm or cancer). Examples of effective thrombolytic agents are streptokinase and urokinase.

Exemplary therapeutic agents include, but are not limited to, antibiotics, antiproliferative agents, rapamycin macrolides, analgesics, anesthetics, antiangiogenic agents, vasoactive agents, anticoagulants, immunomodulators, cytotoxic agents, antiviral agents, antithrombotic drugs, antibodies, neurotransmitters, psychoactive drugs, and combinations thereof. Additional examples of therapeutic agents include, but are not limited to, cell cycle control agents; agents which inhibit cyclin protein production; cytokines, including, but not limited to, Interleukins 1 through 13 and tumor necrosis factors; anticoagulants, anti-platelet agents; TNF receptor domains and the like. Typically the therapeutic agent is neutral or positively charged. In certain instances, where the therapeutic agent is negatively charged, an additional charge neutralization moiety (e.g., a cationic peptide) can be used.

Effective anticellular agents include classical chemotherapeutic agents, such as steroids, antimetabolites, anthracycline, vinca alkaloids, antibiotics, alkylating agents, epipodophyllotoxin and anti-tumor agents such as neocarzinostatin (NCS), adriamycin and dideoxycytidine; mammalian cell cytotoxins, such as interferon-α (IFN-α), interferon-βγ (IFN-βγ), interleukin-12 (IL-12) and tumor necrosis factor-α (TNF-α); plant-, fungus- and bacteria-derived toxins, such as ribosome inactivating protein, gelonin, α-sarcin, aspergillin, restrictocin, ribonucleases, diphtheria toxin, Pseudomonas exotoxin, bacterial endotoxins, the lipid A moiety of a bacterial endotoxin, ricin A chain, deglycosylated ricin A chain and recombinant ricin A chain; as well as radioisotopes.

As used herein, a cargo molecule can be (1) any heterologous polypeptide, or fragment thereof, (2) any polynucleotide (e.g., a ribozyme. RNAi (siRNA, shRNA etc), antisense molecule, PMO, PNA, polynucleotide, oligonucleotide and the like); (3) any small molecule, or (4) any diagnostic or therapeutic agent, that is capable of being linked or complexed to a dendritic polymer of the disclosure or loaded into a LNP disclosed herein. For example, the cargo domain can comprise any one or more of siRNA/siRNN RNAi triggers, ASOs, PMOs, PNAs, oligonucleotides (e.g., guide RNA (gRNA) or sequence encoding gRNA), CRISPR DNA/RNA editing, mRNA, DNA Vectors, Lipid Nanoparticles, proteins, peptides, large synthetic molecules. Any such cargo domain can be used to treat diseases and disorders recognized in the art including, but not limited to, cancer, inflammation, infection, autoimmune diseases, pain disorders, growth disorders, antiproliferative disorders, stem cell therapies, genetic abnormalities and the like.

The term “therapeutic” is used in a generic sense and includes treating agents, prophylactic agents, and replacement agents. Examples of therapeutic molecules include, but are not limited to, cell cycle control agents; agents which inhibit cyclin proteins, such as antisense polynucleotides to the cyclin G1 and cyclin D1 genes; growth factors such as, for example, epidermal growth factor (EGF), vascular endothelial growth factor (VEGF), erythropoietin, G-CSF, GM-CSF, TGF-α, TGF-β, and fibroblast growth factor; cytokines, including, but not limited to, Interleukins 1 through 13 and tumor necrosis factors; anticoagulants, anti-platelet agents; anti-inflammatory agents; tumor suppressor proteins; clotting factors including Factor VIII and Factor IX, protein S, protein C, antithrombin III, von Willebrand Factor, cystic fibrosis transmembrane conductance regulator (CFTR), and negative selective markers such as Herpes Simplex Virus thymidine kinase.

In addition, a cargo molecule can be a negative selective marker or “suicide” protein, such as, for example, the Herpes Simplex Virus thymidine kinase (TK) or cytosine deaminase (CD). Such a hydrophilic masked cationic charge dendrimer or hydrophilic masked cationic charge LNP linked to a suicide protein may be administered to a subject whereby tumor cells are selectively transduced. After the tumor cells are transduced with the kinase, an interaction agent, such as gancyclovir or acyclovir or 5-flurocytosine (5-FC), is administered to the subject, whereby the transduced tumor cells are killed.

In addition, a cargo molecule can be a diagnostic agent such as an imaging agent. Exemplary diagnostic agents include, but are not limited to, imaging agents, such as those that are used in positron emission tomography (PET), computer assisted tomography (CAT), single photon emission computerized tomography, X-ray, fluoroscopy, and magnetic resonance imaging (MRI). Suitable materials for use as contrast agents in MRI include, but are not limited to, gadolinium chelates, as well as iron, magnesium, manganese, copper, and chromium chelates. Examples of materials useful for CAT and X-rays include, but are not limited to, iodine based materials.

Examples of radioimaging agents emitting radiation (detectable radio-labels) that may be suitable are exemplified by indium-111, technitium-99, or low dose iodine-131. Detectable labels, or markers, for use in conjunction with or attached to the nucleic acid constructs of the disclosure as auxiliary moieties may be a radiolabel, a fluorescent label, a nuclear magnetic resonance active label, a luminescent label, a chromophore label, a positron emitting isotope for PET scanner, a chemiluminescence label, or an enzymatic label. Fluorescent labels include, but are not limited to, green fluorescent protein (GFP), fluorescein, and rhodamine. The label may be for example a medical isotope, such as for example and without limitation, technetium-99, iodine-123 and-131, thallium-201, gallium-67, fluorine-18, indium-111, etc.

Thus, it is to be understood that the disclosure is not to be limited to any particular cargo domain used for diagnosis and/or treatment of any particular disease or disorder. Rather, the cargo molecule can be any molecule or agent known or used in the art for treatment or diagnostics of a disease or disorder.

When the cargo molecule is a polypeptide, the polypeptide can comprise L-optical isomer or the D-optical isomer of amino acids or a combination of both. Polypeptides that can be used in the disclosure include modified sequences such as glycoproteins, retro-inverso polypeptides, D-amino acid modified polypeptides, and the like. A polypeptide includes naturally occurring proteins, as well as those which are recombinantly or synthetically synthesized. “Fragments” are a portion of a polypeptide. The term “fragment” refers to a portion of a polypeptide which exhibits at least one useful epitope or functional domain. The term “functional fragment” refers to fragments of a polypeptide that retain an activity of the polypeptide. Functional fragments can vary in size from a polypeptide fragment as small as an epitope capable of binding an antibody molecule, to a large polypeptide capable of participating in the characteristic induction or programming of phenotypic changes within a cell. An “epitope” is a region of a polypeptide capable of binding an immunoglobulin generated in response to contact with an antigen. Small epitopes of receptor ligands can be useful in the methods of the invention so long as it retains the ability to interact with the receptor.

In some embodiments, retro-inverso peptides are used. “Retro-inverso” means an amino-carboxy inversion as well as enantiomeric change in one or more amino acids (i.e., levantory (L) to dextrorotary (D)). A polypeptide encompasses, for example, amino-carboxy inversions of the amino acid sequence, amino-carboxy inversions containing one or more D-amino acids, and non-inverted sequence containing one or more D-amino acids. Retro-inverso peptidomimetics that are stable and retain bioactivity can be devised as described by Brugidou et al. (Biochem. Biophys. Res. Comm. 214(2): 685-693, 1995) and Chorev et al. (Trends Biotechnol. 13(10): 438-445, 1995).

The methods of the disclosure allow for controlling the optimal number of monomer units making up a cationic charge dendritic polymer as well as the ability to incorporate either a single type of monomer or a variety of different types monomers to generate structurally well-defined diverse cationic charge dendrimer libraries capable of optimizing endosomal escape and delivery of a wide variety of a given type of cargo molecule.

In certain embodiments, the hydrophilic masked cationic charge dendrimer or the hydrophilic masked cationic charge LNP may further include a targeting moiety. The disclosure provides for one or more targeting moieties which can be attached to compound disclosed herein as an auxiliary moiety, for example as a targeting auxiliary moiety. A targeting moiety is selected based on its ability to target a compound disclosed herein to a desired or selected cell population that expresses the corresponding binding partner (e.g., either the corresponding receptor or ligand) for the selected targeting moiety. A targeting moiety is also selected based upon its ability to either induce endocystosis or attach to a cell surface protein that endocytoses. For example, a compound of the disclosure could be targeted to cells expressing epidermal growth factor receptor (EGFR) by selected epidermal growth factor (EGF) as the targeting moiety that induces endocytosis.

In one embodiment, the targeting moiety is a receptor binding domain. In another embodiment, the targeting moiety is or specifically binds to a protein selected from the group comprising insulin, insulin-like growth factor receptor 1 (IGF1R), IGF2R, insulin-like growth factor (IGF; e.g., IGF 1 or 2), mesenchymal epithelial transition factor receptor (c-met; also known as hepatocyte growth factor receptor (HGFR)), hepatocyte growth factor (HGF), epidermal growth factor receptor (EGFR), epidermal growth factor (EGF), heregulin, fibroblast growth factor receptor (FGFR), platelet-derived growth factor receptor (PDGFR), platelet-derived growth factor (PDGF), vascular endothelial growth factor receptor (VEGFR), vascular endothelial growth factor (VEGF), tumor necrosis factor receptor (TNFR), tumor necrosis factor alpha (TNF-α), TNF-β, folate receptor (FOLR), folate, transferring, transferrin receptor (TfR), mesothelin, Fc receptor, c-kit receptor, c-kit, an integrin (e.g., an α4 integrin or a ß-1 integrin). P-selectin, sphingosine-1-phosphate receptor-1 (SIPR), hyaluronate receptor, leukocyte function antigen-1 (LFA-1), CD4, CD11, CD18, CD20, CD25, CD27,CD52, CD70, CD80, CD85, CD95 (Fas receptor), CD106 (vascular cell adhesion molecule 1 (VCAM4), CD166 (activated leukocyte cell adhesion molecule (ALCAM)), CD178 (Fas ligand), CD253 (TNF-related apoptosis-inducing ligand (TRAIL)), ICOS ligand, CCR2, CXCR3, CCR5, CXCL12 (stromal cell-derived factor 1 (SDF-1)), interleukin 1 (IL-1), IL-1ra, IL-2, IL-3, IL-4, IL-6, IL-7, IL-8, CTLA-4, MART-1, gp100, MAGE-1, ephrin (Eph) receptor, mucosal addressin cell adhesion molecule 1 (MAdCAM-1), carcinoembryonic antigen (CEA), Lewis^{Y}, MUC-1, epithelial cell adhesion molecule (EpCAM), cancer antigen 125 (CA125), prostate specific membrane antigen (PSMA), TAG-72 antigen, and fragments thereof. In a further embodiment, the targeting moiety is erythroblastic leukemia viral oncogene homolog (ErbB) receptor (e.g., ErbB1 receptor; ErbB2 receptor; ErbB3 receptor; and ErbB4 receptor).

The targeting moiety can also be selected from bombesin, gastrin, gastrin-releasing peptide, tumor growth factors (TGF), such as TGF-α and TGF-β, and vaccinia virus growth factor (VVGF). Non-peptidyl ligands can also be used as the targeting moiety and may include, for example, steroids, carbohydrates, vitamins, and lectins. The targeting moiety may also be selected from a peptide or polypeptide, such as somatostatin (e.g., a somatostatin having the core sequence cyclo[Cys-Phe-D-Trp-Lys-Thr-Cys], and in which, for example, the C-terminus of the somatostatin analog is: Thr-NH₂), a somatostatin analog (e.g., octreotide and lanreotide), bombesin, a bombesin analog, or an antibody, such as a monoclonal antibody.

Other peptides or polypeptides for use as a targeting moiety in the hydrophilic masked cationic charge dendrimer or the hydrophilic masked cationic charge LNP of the disclosure can be selected from KiSS peptides and analogs, urotensin II peptides and analogs. GnRH I and II peptides and analogs, depreotide, vapreotide, vasoactive intestinal peptide (VIP), cholecystokinin (CCK), RGD-containing peptides, melanocyte-stimulating hormone (MSH) peptide, neurotensin, calcitonin, peptides from complementarity determining regions of an antitumor antibody, glutathione, YIGSR (leukocyte-avid peptides, e.g., P483H, which contains the heparin-binding region of platelet factor-4 (PF-4) and a lysine-rich sequence), atrial natriuretic peptide (ANP), β-amyloid peptides, delta-opioid antagonists (such as ITIPP (psi)), annexin-V, endothelin, leukotriene B4 (LTB4), chemotactic peptides (e.g., N-formyl-methionyl-leucyl-phenylalanine-lysine (fMLFK)), GP IIb/IIIa receptor antagonists (e.g., DMP444), human neutrophil elastase inhibitor (EPI-HNE-2 and EPI-HNE-4), plasmin inhibitor, antimicrobial peptides, apticide (P280) and P274), thrombospondin receptor (including analogs such as TP-1300), bitistatin, pituitary adenylyl cyclase type I receptor (PAC1), fibrin α-chain, peptides derived from phage display libraries, and conservative substitutions thereof.

Immunoreactive ligands for use as a targeting moiety in the hydrophilic masked cationic charge dendrimer or the hydrophilic masked cationic charge LNP of the disclosure include an antigen-recognizing immunoglobulin (also referred to as “antibody”), or antigen-recognizing fragment thereof that is capable of inducing endocystosis. As used herein, “immunoglobulin” refers to any recognized class or subclass of immunoglobulins such as IgG, IgA, IgM, IgD, or IgE. Typical are those immunoglobulins which fall within the IgG class of immunoglobulins. The immunoglobulin can be derived from any species. Typically, however, the immunoglobulin is of human, murine, or rabbit origin. In addition, the immunoglobulin may be polyclonal or monoclonal, but is typically monoclonal.

Targeting moieties of the disclosure may include an antigen-recognizing immunoglobulin fragment. Such immunoglobulin fragments may include, for example, the Fab′, F(ab′)₂, Fᵥ or Fab fragments, single-domain antibody, ScFv, or other antigen-recognizing immunoglobulin fragments. Fc fragments may also be employed as targeting moieties. Such immunoglobulin fragments can be prepared, for example, by proteolytic enzyme digestion, for example, by pepsin or papain digestion, reductive alkylation, or recombinant techniques. The materials and methods for preparing such immunoglobulin fragments are well-known to those skilled in the art. See Parham, J. Immunology, 131, 2895, 1983; Lamovi et al., J. Immunological Methods, 56, 235, 1983.

Targeting moieties of the disclosure include those targeting moieties which are known in the art but have not been provided as a particular example in this disclosure that either induce endocytosis or are endocytosed.

A pharmaceutical composition according to the disclosure can be prepared to include a hydrophilic masked cationic charge dendrimer or a hydrophilic masked cationic charge LNP of the disclosure, into a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton; Mack Publishing Co., 1405-1412, 1461-1487 (1975), and The National Formulary XIV., 14th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The pharmaceutical compositions according to the disclosure may be administered locally or systemically. By “therapeutically effective dose” is meant the quantity of a fusion polypeptide according to the disclosure necessary to prevent, to cure, or at least partially arrest the symptoms of a disease or disorder (e.g., to inhibit cellular proliferation). Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of particular disorders. Various considerations are described, e.g., in Langer, Science, 249:1527, (1990); Gilman et al. (eds.) (1990), each of which is herein incorporated by reference.

As used herein, “administering a therapeutically effective amount” is intended to include methods of giving or applying a pharmaceutical composition of the disclosure to a subject that allow the composition to perform its intended therapeutic function. The therapeutically effective amounts will vary according to factors, such as the degree of disease in a subject, the age, sex, and weight of the individual. Dosage regimen can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, intracerebrally, intraspinal, intraocular, and the like), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically, the composition will be stable under the conditions of manufacture and storage and preserved against the contaminating action of microorganisms, such as bacteria and fungi. The carrier can be a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the subject's diet. For oral therapeutic administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit.

The tablets, troches, pills, capsules, and the like can also contain the following; a binder, such as gum gragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic in the amounts employed. In addition, the pharmaceutical composition can be incorporated into sustained-release preparations and formulations.

Thus, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

It is especially advantageous to formulate parenteral compositions in dosage unit form for ease of administration and uniformity of dosage. “Dosage unit form” as used herein, refers to physically discrete units suited as unitary dosages for the subject to be treated; each unit containing a predetermined quantity of pharmaceutical composition is calculated to produce the desired therapeutic effect in association with the required pharmaceutical carrier. The specification for the dosage unit forms of the disclosure are related to the characteristics of the pharmaceutical composition and the particular therapeutic effect to be achieve.

The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

The working examples below are provided to illustrate, not limit, the invention. Various parameters of the scientific methods employed in these examples are described in detail below and provide guidance for practicing the invention in general.

### EXAMPLES

Synthesis of a hydrophilic masked cationic charge dendrimers. The hydrophilic masked cationic charge dendrimers were synthesized using the synthetic routes and reactions presented in FIGS. 2, 3, 5, 7, and 9.

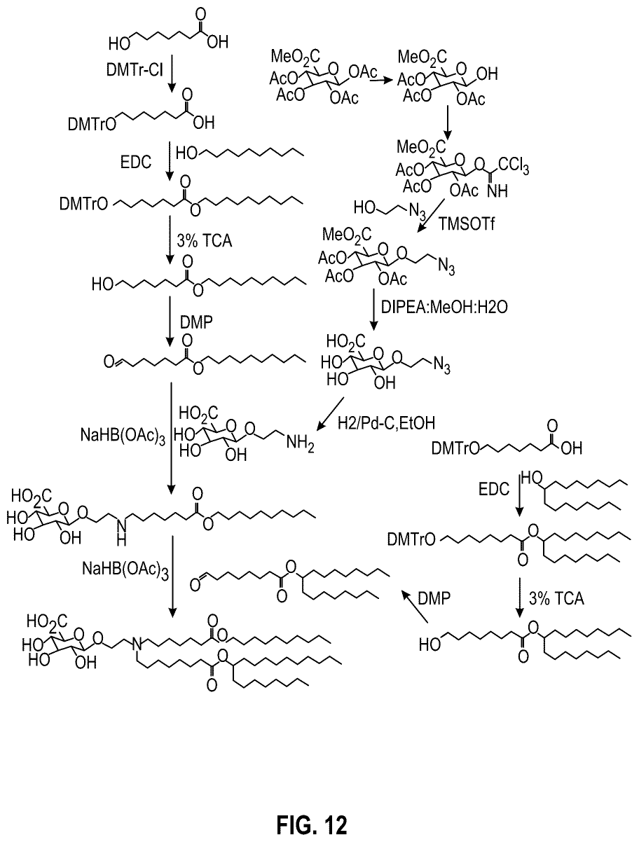

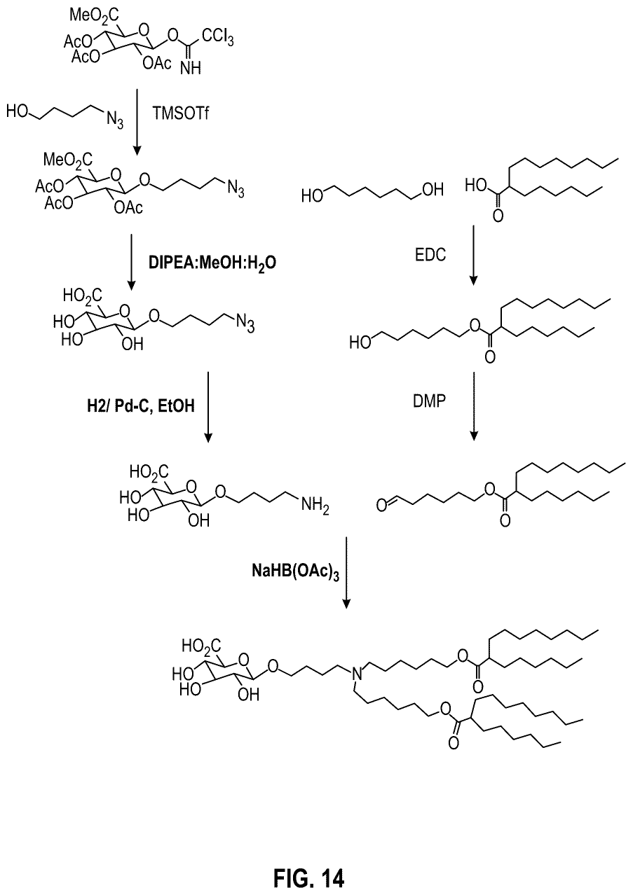

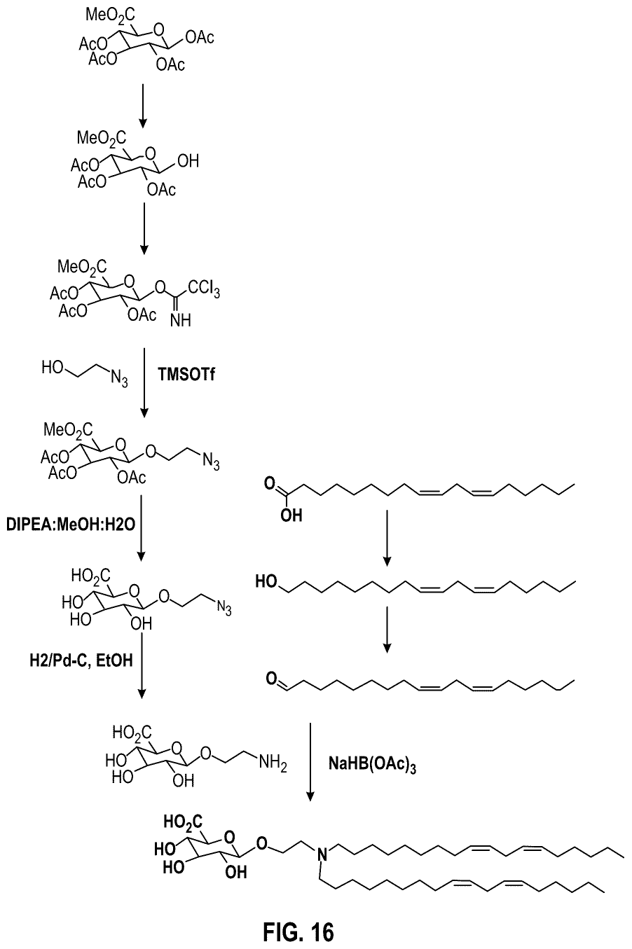

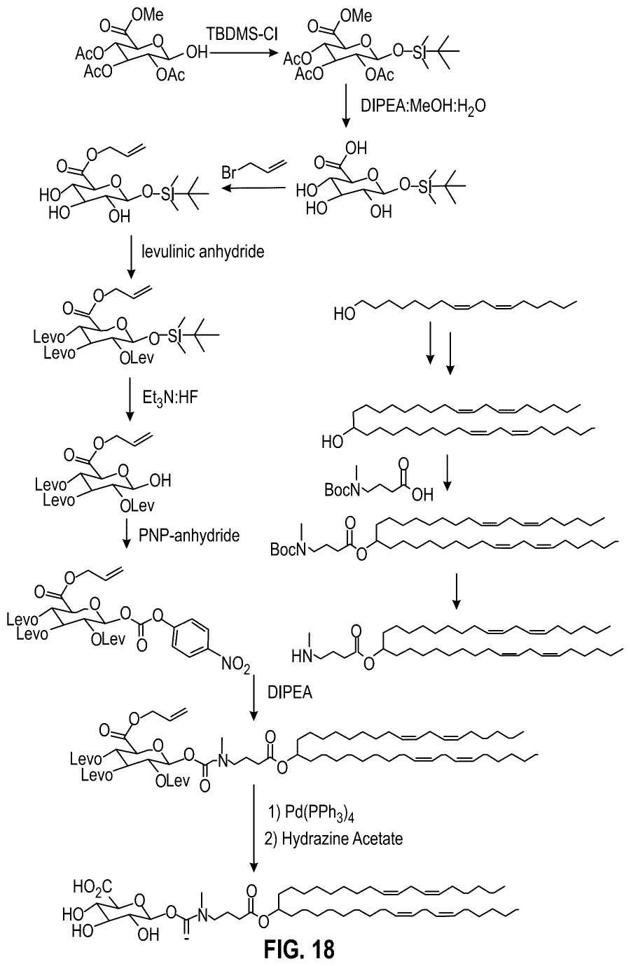

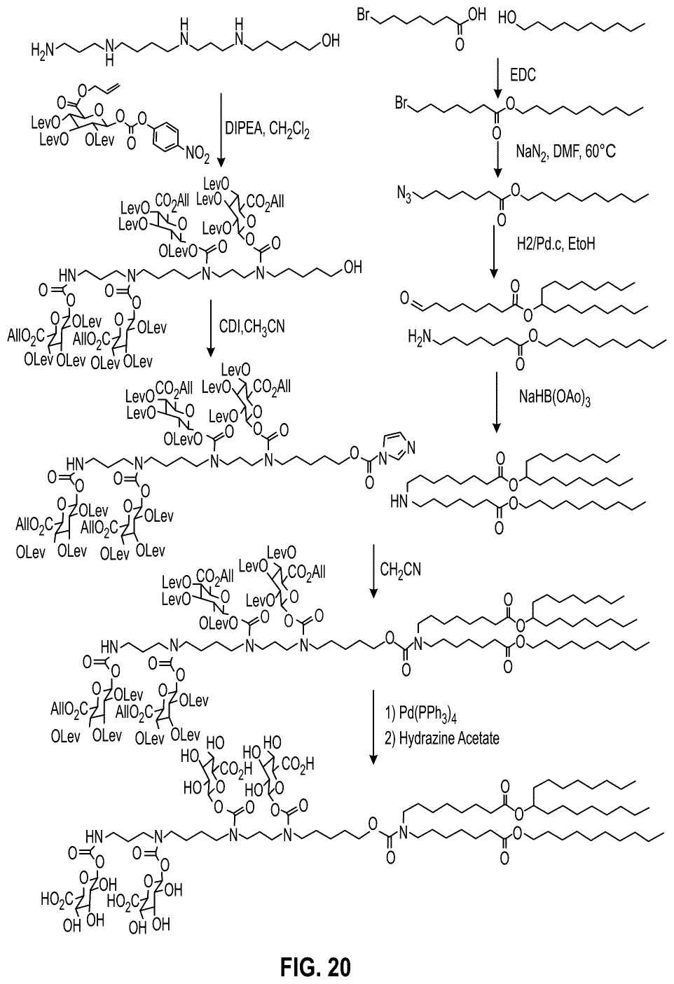

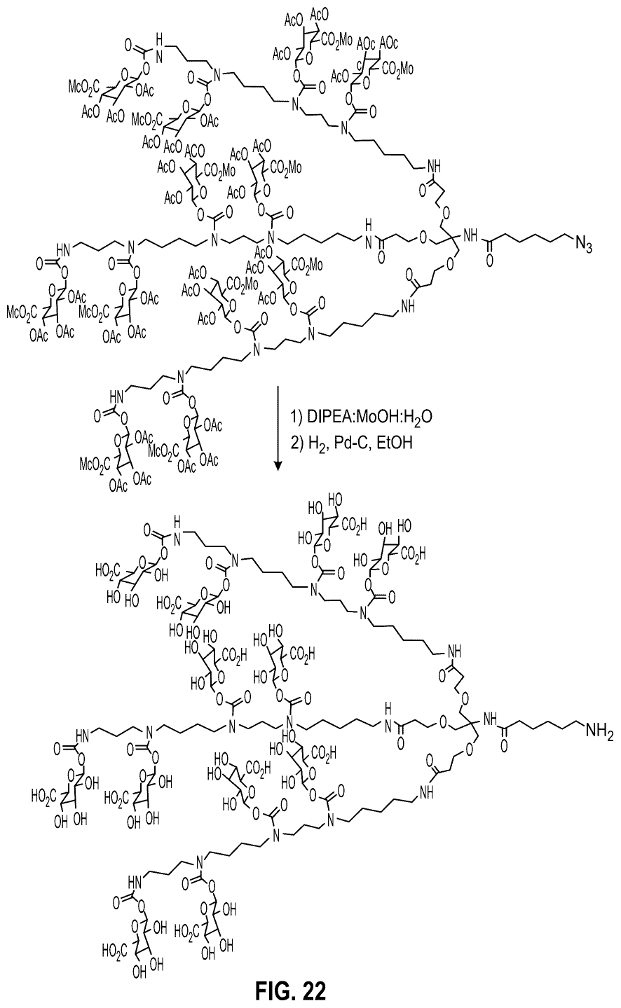

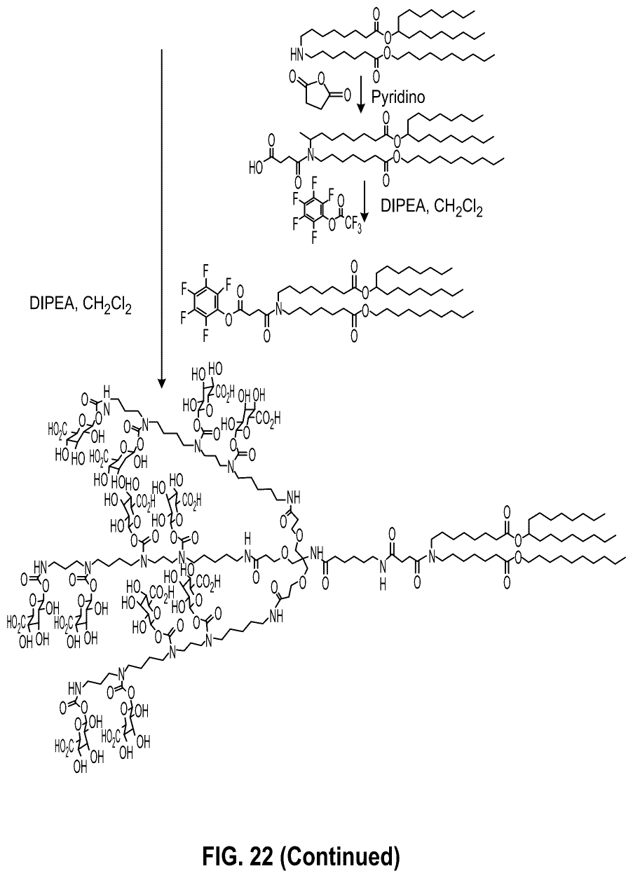

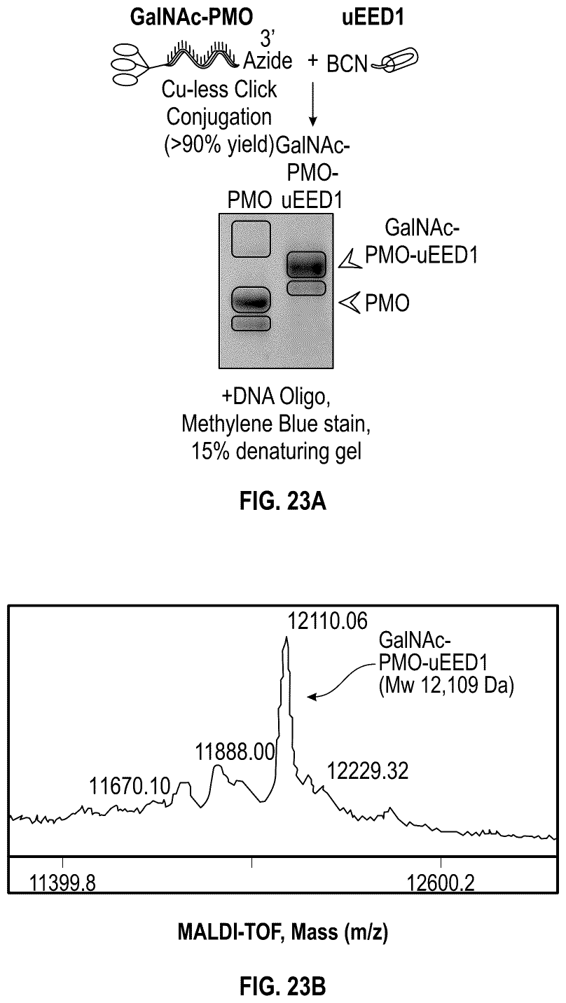

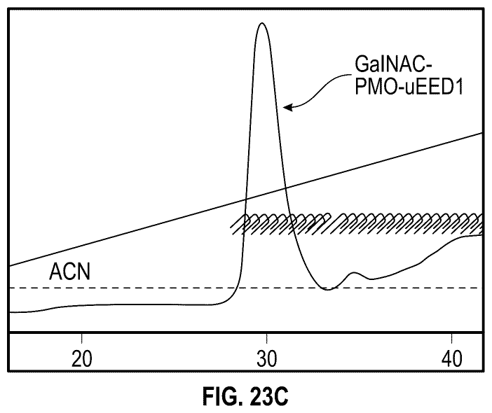

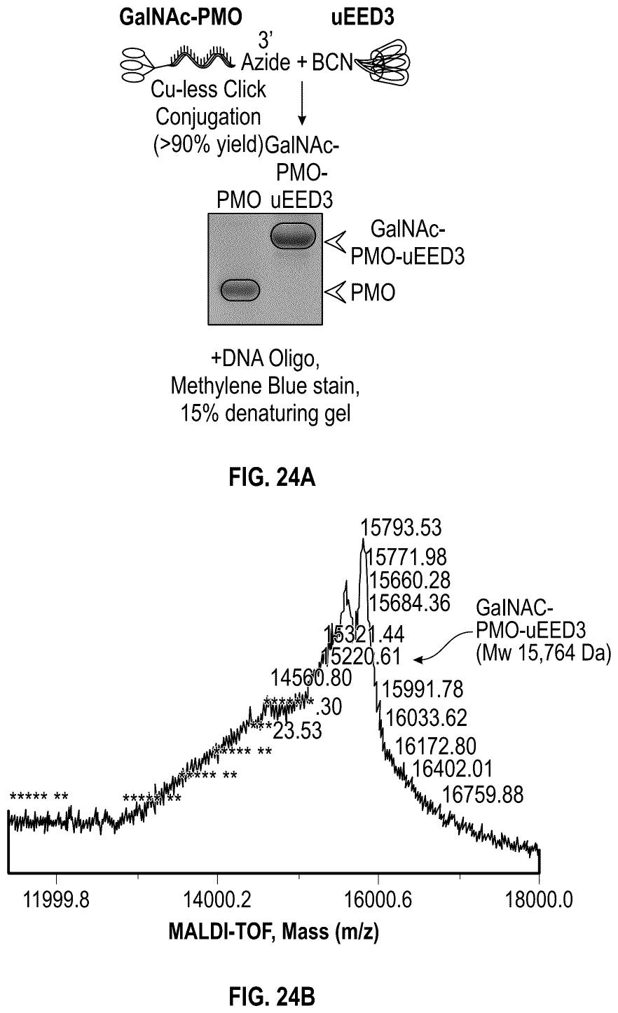

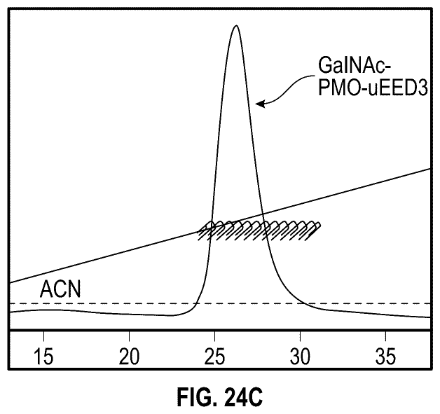

Synthesis of a hydrophilic masked cationic charge LNPs. The hydrophilic masked cationic charge LNPs were synthesized using the synthetic routes and reactions presented in FIGS. 12, 14, 16, 18, 20 and 22.

Hydroxypentyl amine-Boc-Spermine. To a solution of Mono-amine-Boc-Spermine, in dichloroethane at rt was added 5-hydroxypentanal, NaHB(OAc)₃ (1.5 eq). The reaction mixture was stirred for 18 h at rt. Crude mixture was directly loaded on silica-gel and purified by combiflash chromatography using 0 to 20% MeOH (10% NH₄OH) CH₂Cl₂ to obtain pure product.

Hydroxypentyl-Boc-Spermine. To a solution of Hydroxypentyl amine-Boc-Spermine, in MeOH, Boc anhydride, NEt₃, were added at 0° C. and then stirred at rt for 18 h. Evaporated, and crude mixture was directly loaded on silica-gel and purified by combiflash chromatography using 0 to 10% MeOH/CH₂Cl₂ to obtain pure product.

Mesylpentyl-Boc-Spermine. To a solution of Hydroxypentyl-Boc-Spermine, NEt₃, in CH₂Cl₂, solution of Mesylchloride in CH₂Cl₂, were dropwise added at 0° C. and then stirred at rt for 3 h. Reaction mixture was diluted with CH₂Cl₂ and washed with saturated Bicarbonate solution, dried, evaporated, and crude mixture purified by combiflash chromatography using 0 to 10% MeOH/CH₂Cl₂ to obtain pure product.

Azidepentyl-Boc-Spermine. Mesylpentyl-Boc-Spermine was dissolved in DMF. To the solution, sodium azide was added. The reaction was stirred at 65° C, for overnight. The solvent was removed under reduced pressure and the residue was purified by silica gel column chromatography (0 % to 10% MeOH/CH₂Cl₂) to produce a thick liquid.

Aminopentyl-Boc-Spermine. To a mixture of Azidepentyl-Boc-Spermine dissolved in Dioxane: H₂O (4:1, v/v) was added a 1M Me₃P solution in Toluene, and the reaction mixture was stirred at rt for 4 h. Ammonium hydroxide (30%) was added to the reaction mixture and the reaction was allowed to stir at rt for 18 h. The mixture was evaporated under reduced pressure and co-evaporated with acetonitrile. The crude product was used in the next reaction as it is.

Acetylated Glucuronide-Spermine-Tris-hexylazide. To a stirred solution of Free spermine-Tris-hexylazide in DMF, solution of AcetateGlucuronide-p-nitrophenylcarbonate, CH₂Cl₂ and DIPEA were added, and the reaction mixture was allowed to be stirred at rt for 18 h. Mixture was diluted with CH₂Cl₂ and washed with brine (5×). dried over Na₂SO₄, filtered, and concentrated under reduced pressure. The residue obtained was purified by flash column (silica-gel) chromatography and eluted with 0% to 20% EtOAc/CH₂Cl₂ to yield the product.

Acetylated Glucuronide-Spermine-hexylamine. Acetylated Glucuronide-Spermine-hexylazide was dissolved in EtOH:CH₂Cl₂, into solution 10% Pd/C was added, reaction carried out under hydrogen balloon (1 atm) condition and allowed to be stirred for 18 h at rt under hydrogen. Reaction mixture was filtered through celite pad and washed with methanol. Evaporated to dryness and crude product was used in next step without purification.

Glucuronide-Spermine-Bicyclononyne. Acetylated Glucuronide-Spermine-hexylamine was reacted with (1R,8S,9s)-Bicyclo[6.1.0]non-4-yn-9-ylmethyl Succinimidyl Carbonate for 2 h. The crude material was purified on combiflash. Obtained compound was treated with DIPEA: MeOH: H₂O. (0.5:5:1, v/v/v) at rt for 18 h. Mixture was evaporated to dryness by lyophilization.

Boc-Spermine-Tris-hexylazide. To a stirred solution of acid pentaflurophenyl ester-Tris-hexylazide in CH₂Cl₂, Boc-spermine-amine, and DIPEA were added, and the reaction mixture was allowed to be stirred at rt for 18 h. Concentrated under reduced pressure. The residue obtained was purified by flash column (silica-gel) chromatography and eluted with 0% to 10% MeOH/CH₂Cl₂ to yield product.

Free-Spermine-Tris-hexylazide. To a mixture of Boc-Spermine-Tris-hexylazide in CH₂Cl₂ was treated with 4M HCl/Dioxane). Mixture stirred for 18 h at rt. Evaporated to dryness on rotavapor at 45° C. Co-evaporation with CH₃CN (4×). White solids (salts) were treated with 19N NaOH aq, extracted with CHCl₃. Dried over anhydrous Na₂SO₄ and evaporated to obtain crude compound.

Acetylated Glucuronide-Spermine-Tris-hexylazide. To a stirred solution of Free spermine-Tris-hexylazide in DMF, solution of AcetateGlucuronide-p-nitrophenylcarbonate. CH₂Cl₂ and DIPEA were added, and the reaction mixture was allowed to be stirred at rt for 18 h. Mixture was diluted with CH₂Cl₂ and washed with brine (5×), dried over Na₂SO₄, filtered, and concentrated under reduced pressure. The residue obtained was purified by flash column (silica-gel) chromatography and eluted with 0% to 20% EtOAc/CH₂Cl₂ to yield the product.

Acetylated Glucuronide-Spermine-Tris-hexylamine. Acetylated Glucuronide-Spermine-Tris-hexylazide was dissolved in EtOH:CH₂Cl₂, into solution 10% Pd/C was added, reaction carried out under hydrogen balloon (1 atm) condition and allowed to be stirred for 18 h at rt under hydrogen. Reaction mixture was filtered through celite pad and washed with methanol. Evaporated to dryness and crude product was used in next step without purification. To the mixture of acetylated glucuronide-spermine-Tris-hexylamine, and dioctadecyl Pentafluorophenyl succinate in dichloromethane, DIPEA was added. The mixture was stirred for 18 h at rt. The crude material was purified on combiflash. Obtained compound was treated with DIPEA:MeOH: H₂O. (0.5:5:1, v/v/v) at rt for 18 h. Mixture was evaporated to dryness by lyophilization.

Nonamer-Glucuronide-Spermine-Oligonucleotide. Nonamer-Azide-Pentaflurophenyl ester was add at 2.5:1 ratio to amino oligonucleotide in sodium tetraborate (pH 8.5) for 18 h at rt. The reaction was purified by C18 HPLC, fractions pooled and lyophilized. Product was brought up in 50%/50% water/acetonitrile and reacted with Glucuronide-Spermine-Bicyclononyne at a 1:2.5 ratio for 18 h at rt. The reaction was purified by C18 HPLC, fractions pooled and lyophilized.

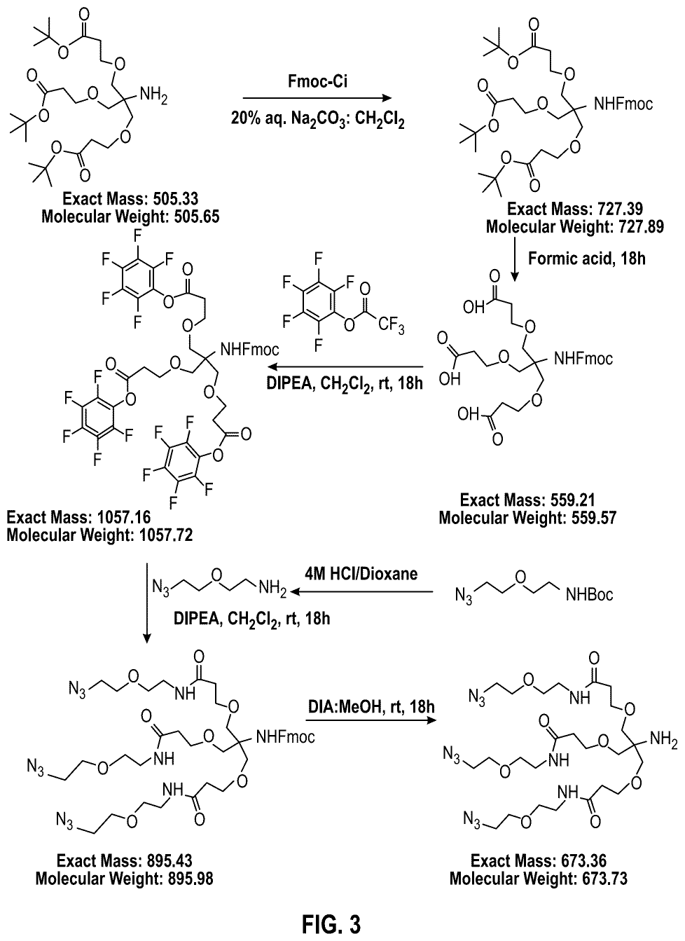

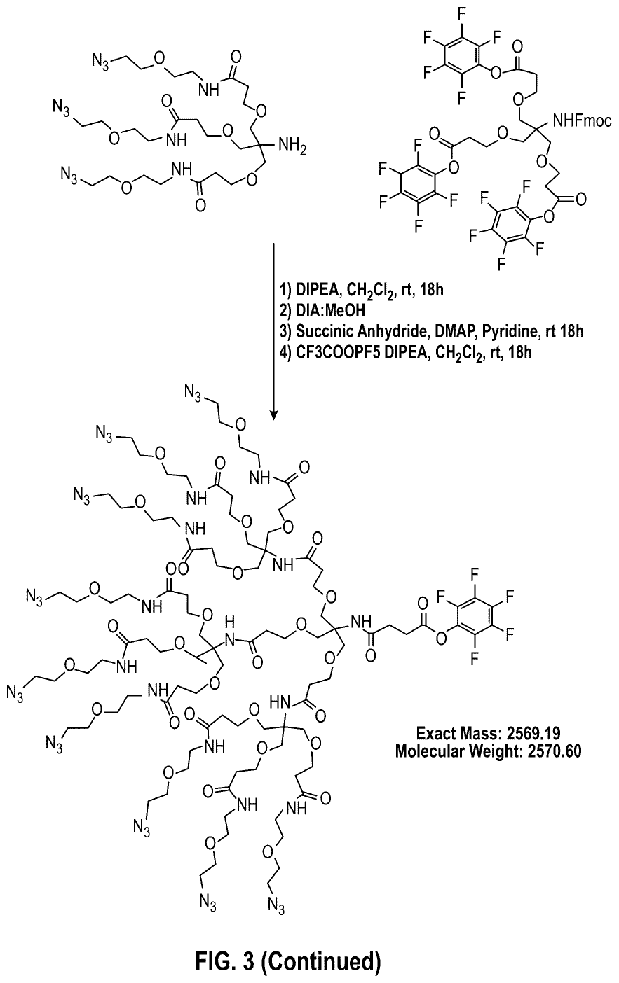

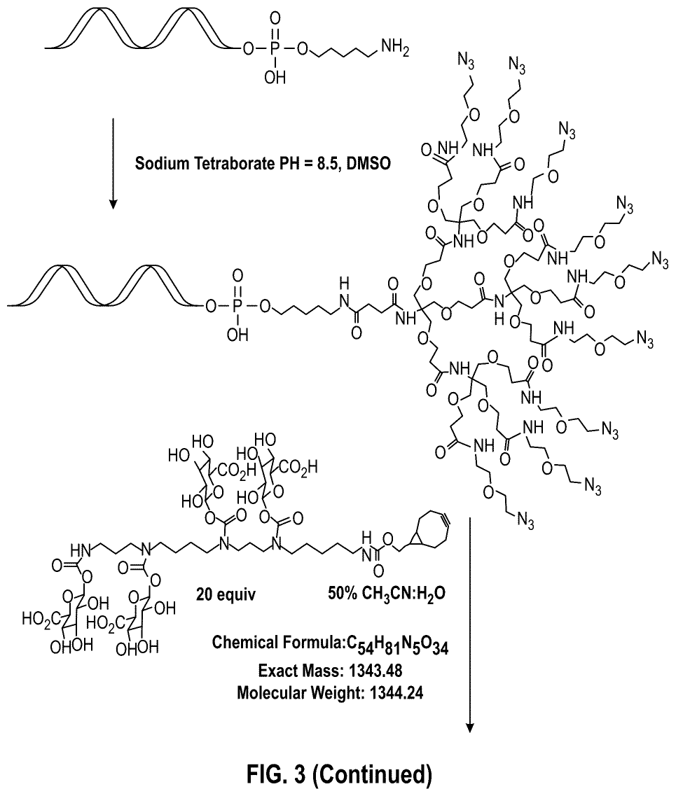

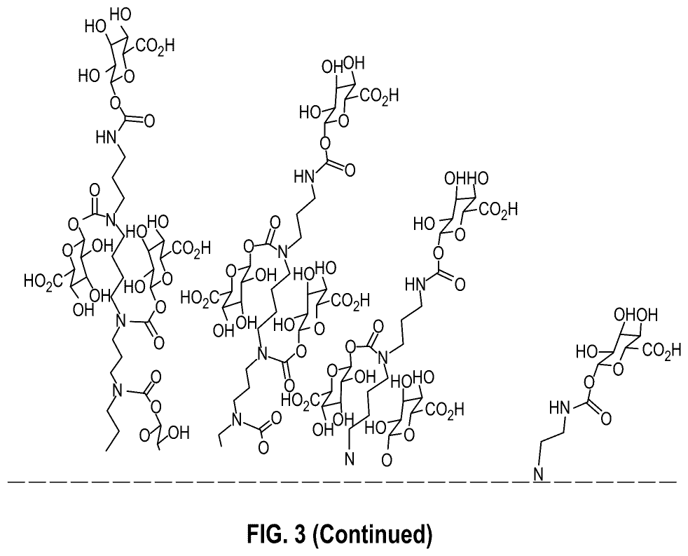

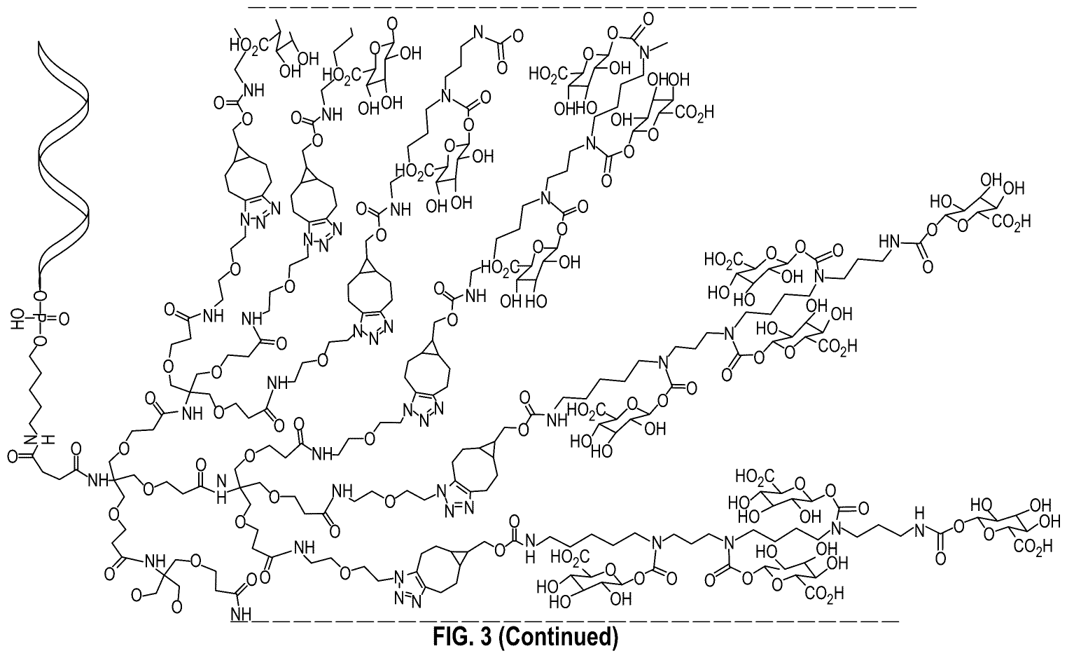

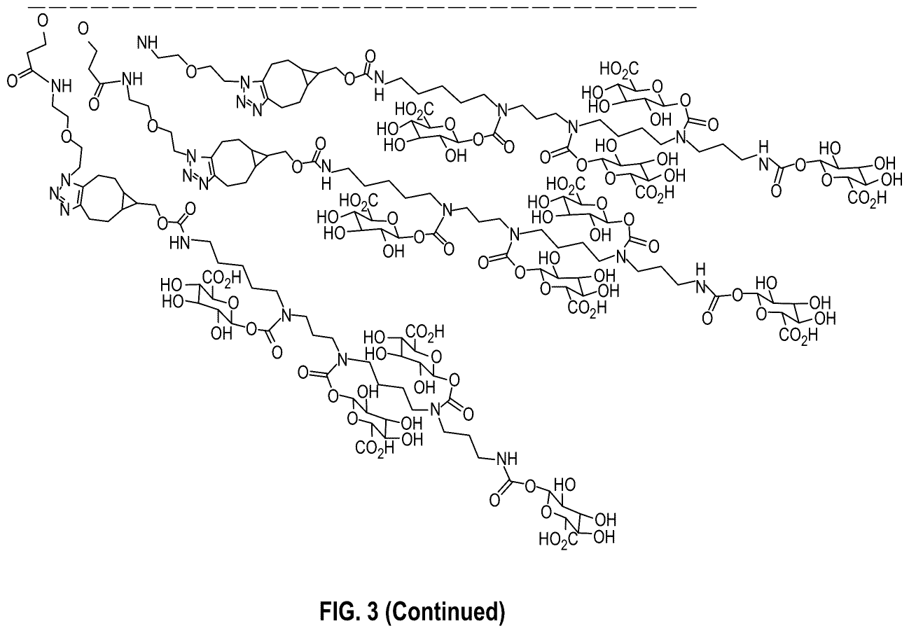

Similar procedures were used and combined for FIGS. 3, 4, 5, 7 and 9.

Synthesis of Hemiacetal Glucuronate. To a solution of Peracetate glucuronate Methylester (Crude) in THF (60 ml) was added Tributyltin methoxide dropwise. The mixture was heated at 70° C, for 1.5 h then solution was allowed to warm to room temperature. The reaction was concentrated under reduced pressure. Crude product was purified (two times) by combiflash chromatography using 0 to 10% MeOH/CH₂Cl₂ to obtain dark brown syrup.

Synthesis of trichloroacetimido (TCA)-Glucuronate. Methyl 2,3,4-triacetyl-a,b-glucopyranuronate 6 (2.98 g, 8.9 mmol) and trichloroacetonitrile (was stirred in dry CH₂Cl₂ at 0° C, for 30 min. DBU was added dropwise and the solution was allowed warm to room temperature and stirred overnight. The solvent was removed in vacuo and the residue was subjected to flash chromatography (40:59:1 EtOAc-hexane-Et₃N). Appropriate fractions were pooled, and the solvent was removed under reduced pressure to yield a syrup to yield product.

Synthesis of Chloropropyl Glucuronate. To the mixture of and MS 4Å, dichloromethane was added, followed by the addition of 3-chloropropanol. The mixture was stirred for 30 min at room temperature under Ar. After being cooled down to 0° C., boron trifluoride ether complex was added in a drop-wise manner. The reaction was stirred at 0° C. for 3 h. After the TLC showed the completion of the reaction, the mixture was filtered, and the filtrate was washed with saturated NaHCO3. The organic layer was evapored to produce a crude residue which was purified by silica gel column chromatography (CH₂Cl₂:EtOAc=4:1 by volume) to provide product as a white solid.

Synthesis of Azidopropyl Glucuronate. Methyl 2,3,4-tetra-O-acetyl-1-O-(3-chloropropyl)-D-glucopyranuronate was dissolved in 20 mL of DMF. To the solution, sodium azide was added. The reaction was stirred at 65° C. for overnight. The solvent was removed under reduced pressure and the residue was purified by silica gel column chromatography (CH₂Cl₂:EtOAc=4:1 by volume) to produce as a white solid.

Synthesis of Aminopropyl Glucuronate. To a stirred mixture of Azidopropyl Glucuronate in EtOH, EtOAc, 10% Pd-C, and Acetic acid was added, and reaction was performed under hydrogen gas balloon at room temperature over 3 h. Reaction mixture was filtered over celite pad and filtrate was evaporated to dryness, co-evaporated with dry acetonitrile (2×). Obtained crude product was used without purification in next step.

Synthesis of Acetyl Glucuronate-315 lipid. To a solution of 315-aldehyde, in dichloroethane at rt was added Aminopropyl Glucuronate. NaHB(OAc)₃, and AcOH. The reaction mixture was stirred for 18 h at rt. Crude mixture was directly loaded on silica-gel and purified by combiflash chromatography using 0 to 10% MeOH/CH₂Cl₂ to pure acetyl Glc-315 lipid.

Synthesis of Glc-315 lipid. Acetyl Glucuronate-315 lipid was dissolved in (DIPEA: MeOH: H₂O 1:10:2, v/v/v) and dichloroethane at rt. The reaction mixture was stirred for 18h+18h+18h at rt and reaction progress was monitored by MALDI-MS. Crude mixture was directly evaporated to dryness, co-evaporated with dry acetonitrile (3×) to obtain Glc-MC lipid.

Similar processes outlined above were combined and used for FIGS. 12, 14, 16, 18, 20 and 22.

Delivery of RNA Oligonucleotides using hydrophilic masked cationic charge dendrimers into Mammalian Cells. To test the ability the hydrophilic masked cationic charge dendrimers to enhance endosomal escape and hence delivery of RNA oligonucleotide therapeutics into cells in vitro, various sized cationic charge dendrimers were conjugated to anti-Luc siRNA molecules via borate catalyzed amide bond formation.

Primary murine hepatocytes from ROSA26-Lox-Stop-lox (LSL) Luciferase mice are pretreated a week (or more) prior with an i.v. administered Adenovirus-Cre to recombine out the LSL DNA segment and thereby constitutively express Luciferase, are isolated per standard protocols and placed into cell culture. Luciferase expressing hepatocytes plated in 24 well plates are treated with various hydrophilic masked cationic charge dendrimer constructs described above and compared to designed control constructs, and untreated hepatocytes. Treated hepatocytes are monitored for RNAi knockdown of Luciferase by plate reader and IVIS imaging assays. All experiments are performed in triplicate and repeated on three independent days (biological triplicates of triplicates). It is expected that various hydrophilic masked cationic charge dendrimer anti-Luc siRNA constructs will result in more efficient RNAi luciferase knockdown and thereby require a lower dose vs. the control constructs.

Delivery of RNA Oligonucleotides using hydrophilic masked cationic charge LNPs into Mammalian Cells. To test the ability the hydrophilic masked cationic charge LNPs to enhance endosomal escape and hence delivery of RNA oligonucleotide therapeutics into cells in vitro, various types of hydrophilic masked cationic charge LNPs were loaded with anti-Luc siRNA molecules.

Primary murine hepatocytes from ROSA26-Lox-Stop-lox (LSL) Luciferase mice are pretreated a week (or more) prior with an i.v. administered Adenovirus-Cre to recombine out the LSL DNA segment and thereby constitutively express Luciferase, are isolated per standard protocols and placed into cell culture. Luciferase expressing hepatocytes plated in 24 well plates are treated with the various hydrophilic masked cationic charge LNPs loaded with anti-Luc siRNA molecules described above and compared to designed control LNPs, and untreated hepatocytes. Treated hepatocytes are monitored for RNAi knockdown of Luciferase by plate reader and IVIS imaging assays. All experiments are performed in triplicate and repeated on three independent days (biological triplicates of triplicates). It is expected that various hydrophilic masked cationic charge LNPs loaded with anti-Luc siRNA molecules will result in more efficient RNAi luciferase knockdown and thereby require a lower dose vs. the control LNPs.

Delivery of RNA Oligonucleotides using hydrophilic masked cationic charge dendrimers into Animal Models. To test the ability of the hydrophilic masked cationic charge dendrimers to enhance endosomal escape and delivery of RNA oligonucleotide therapeutics into tissues in preclinical animal models in vivo. Luciferase expressing preclinical mice are treated with various hydrophilic masked cationic charge dendrimer anti-Luc siRNA constructs vs. controls.

ROSA26-Lox-Stop-lox (LSL) Luciferase mice are pretreated with an i.v. administered adenovirus-Cre to recombine out the LSL DNA segment and thereby constitutively express Luciferase in liver hepatocytes. Treated mice will be monitored daily by live animal IVIS imaging for constitutive baseline luciferase expression starting one-week post Adenovirus-Cre infection. To obtain a baseline measurement for all animals, animals are randomized into groups (n=8/group), injected with luciferin, and assayed by live animal IVIS bioluminescence imaging for three days prior to treatment (days −2, −1, 0)). After imaging on day 0, mice are treated by either subcutaneous or i.v. administered hydrophilic masked cationic charge dendrimer anti-Luc siRNA constructs containing from 9, 18, 27, 36 or more masking monomers described above and compared to matching design control constructs and untreated mice. All animal groups are assaved by live animal IVIS bioluminescence imaging following luciferin injection on days 1, 2, 3, 5, 7, 14, 21, 28 (and longer if necessary).

It is anticipated that various hydrophilic masked cationic charge dendrimer anti-Luc siRNA constructs will result in more efficient RNAi luciferase knockdown and thereby requiring a lower dose vs. control constructs.

Delivery of RNA Oligonucleotides using hydrophilic masked cationic charge LNPs into Animal Models. To test the ability of the hydrophilic masked cationic charge LNPs to enhance endosomal escape and delivery of RNA oligonucleotide therapeutics into tissues in preclinical animal models in vivo. Luciferase expressing preclinical mice are treated with various hydrophilic masked cationic charge LNPs loaded with anti-Luc siRNA vs. controls.

ROSA26-Lox-Stop-lox (LSL) Luciferase mice are pretreated with an i.v. administered adenovirus-Cre to recombine out the LSL DNA segment and thereby constitutively express Luciferase in liver hepatocytes. Treated mice will be monitored daily by live animal IVIS imaging for constitutive baseline luciferase expression starting one-week post Adenovirus-Cre infection. To obtain a baseline measurement for all animals, animals are randomized into groups (n=8/group), injected with luciferin, and assayed by live animal IVIS bioluminescence imaging for three days prior to treatment (days −2, −1, 0). After imaging on day 0, mice are treated by either subcutaneous or i.v. administered hydrophilic masked cationic charge LNPs loaded with anti-Luc siRNA and compared to matching design control LNPs, and untreated mice. All animal groups are assaved by live animal IVIS bioluminescence imaging following luciferin injection on days 1, 2, 3, 5, 7, 14, 21, 28 (and longer if necessary).

It is anticipated that various hydrophilic masked cationic charge LNPs loaded with anti-Luc siRNA will result in more efficient RNAi luciferase knockdown and thereby requiring a lower dose vs. control LNPs.

The figures provide a number of variations of hydrophilic masked cationic charge dendrimers and hydrophilic masked cationic charge LNPs of the disclosure, the constructs are not to be limiting and are exemplary only. Moreover, each construct is explicitly contemplated herein. In addition, a number of synthesis methods are provided in the figures; these methods are exemplary only and are not meant to be limiting.

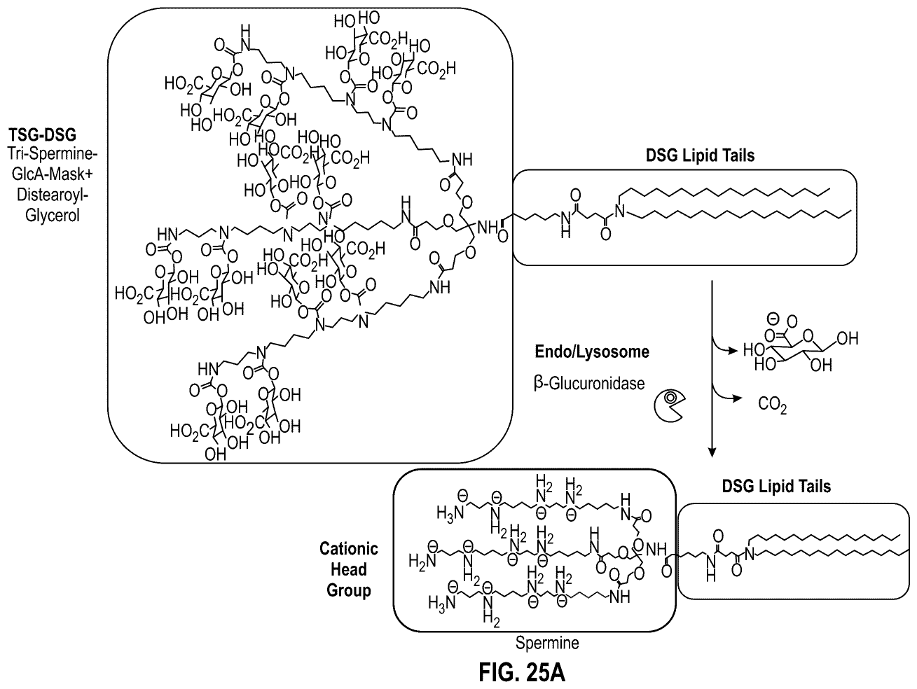

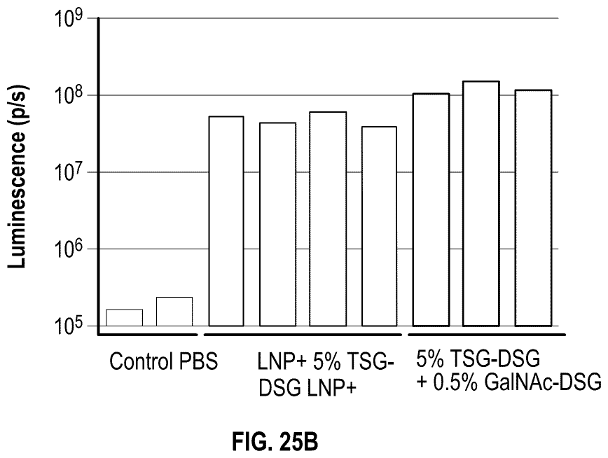

Delivery of mRNA using hydrophilic masked cationic charge LNPs into Animal Models. To test the ability of the hydrophilic masked cationic charge LNPs to enhance endosomal escape and delivery of mRNA therapeutics into tissues in preclinical animal models in vivo, wild type preclinical mice are treated with various hydrophilic masked cationic charge LNPs loaded with Luciferase mRNA vs. controls presented in FIG. 25B.

Mice are treated intraperitoneally with Luciferase mRNA loaded LNPs composed of 50% ALC-0315, 33.5% cholesterol, 10% phosphocholine lipid, 1% PEG-DMG and containing 5% Tris-Spermine-GlcA-Mask+Distearoyl-Glycerol (TSG-DSG) lipid with or without 0.5% GalNAc-PEG-DSG lipid liver hepatocyte targeting domain. At 6-8 h post-treatment mice are analyzed for luciferase expression by injection of luciferin, then whole animal IVIS bioluminescence imaging at 12-20 min (FIG. 25B).

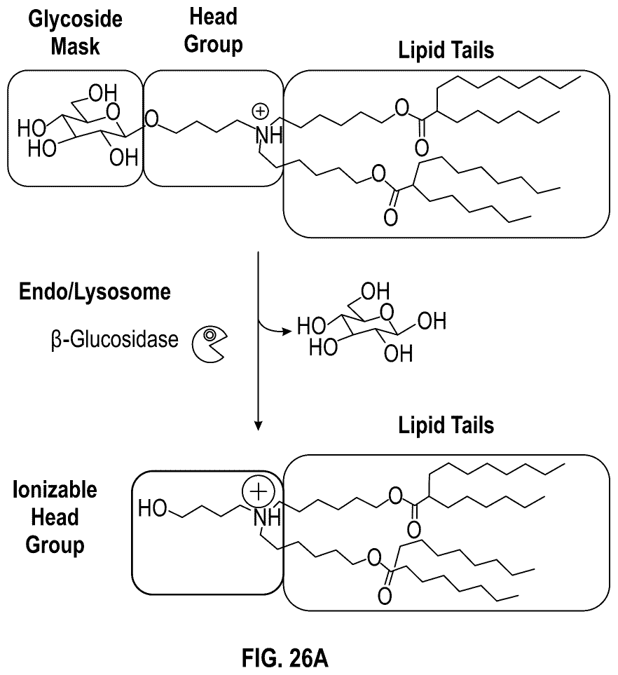

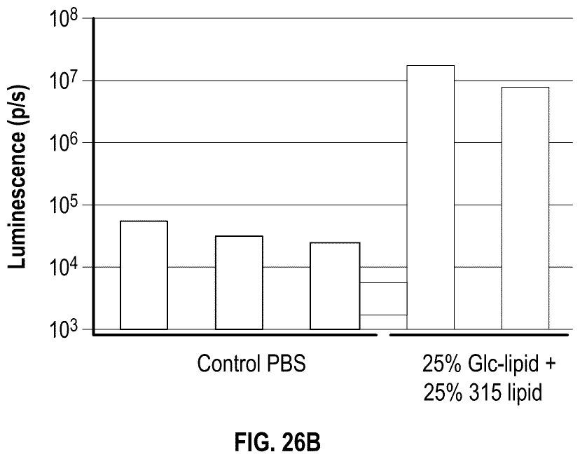

Mice are treated intraperitoneally with Luciferase mRNA loaded LNPs composed of 25% ALC-0315, 39% cholesterol, 10% phosphocholine lipid, 1% PEG-DMG and 25% Glucose (Glc)-Mask-315 lipid. At 6-8 h post-treatment mice are analyzed for luciferase expression by injection of luciferin, then whole animal IVIS bioluminescence imaging at 12-20 min (FIG. 26B).

The figures provide a number of variations of hydrophilic masked cationic charge dendrimers and hydrophilic masked cationic charge NPLs of the disclosure, the constructs are not to be limiting and are exemplary only. Moreover, each construct is explicitly contemplated herein. In addition, a number of synthesis methods are provided in the figures; these methods are exemplary only and are not meant to be limiting.

A number of embodiments have been described. Nevertheless, it will be understood that various modifications may be made without departing from the spirit and scope of the description. Accordingly, other embodiments are within the scope of the following claims.

1. A construct comprising:
(i) at least one cargo domain;
(ii) a dendrimer domain having a first end and a plurality of second ends;
(iii) a plurality of hydrophobic or cationic charge domains;
wherein the first end of the dendrimer domain is linked directly or indirectly to the at least one cargo domain and the plurality of second ends are linked to the plurality of hydrophobic or cationic charge domains.

2-3. (canceled)

4. The construct of claim 1, wherein the plurality of cationic charge or hydrophilic domains are masked by a plurality of hydrophilic mask domains such that the construct has a reduced net cationic, a net neutral or a net anionic charge.

5. (canceled)

6. The construct of claim 1, further comprising a coupler domain between the at least one cargo domain and the first end of the dendrimer domain.

7. The construct of claim 4, wherein the plurality of hydrophilic mask domains each comprise a glycoside moiety.

8. The construct of claim 1, wherein the plurality of hydrophobic domain or cationic charge domain comprise any functional group which contains an aromatic indole ring; nitrogen containing mono-cyclic or multi-cyclic rings; primary, secondary or tertiary amino group; a lipid or a monomeric unit derived therefrom; a tocopherol; a hydrophobic oligomer or a monomeric unit derived therefrom; a hydrophobic polymer or a monomeric unit derived therefrom.

9. The construct of claim 8, wherein the hydrophobic domain comprises a lipid selected from a C8, C10, C12, C14, C16, or C18 lipid or derivative thereof.

10. The construct of claim 8, wherein each of the plurality of hydrophobic domains comprise a monomeric unit derived from a lipid selected from a fatty acid, a fatty alcohol and any other lipid molecule with at least two carbon units.

11. The construct of claim 8, wherein each of the plurality of hydrophobic domains comprise a hydrophobic polymer.

12. The construct of claim 8, wherein each of the plurality of hydrophobic domains comprise one or more monomeric units derived from a hydrophobic polymer selected from the group consisting of: polyester, polyether, polycarbonate, polyanhydride, polyamide, polyacrylate, polymethacrylate, polyacrylamide, polysulfone, polyalkane, polyalkene, polyalkyne, polyanhydride, polyorthoester, N-isopropylacrylamide, methyl acrylate, ethyl acrylate, propyl acrylate, butyl acrylate, methyl methacrylate, ethyl methacrylate, propyl methacrylate, butyl methacrylate, acrylic acid, methacrylic acid, quaternary ammonium-modified acrylate, quaternary ammonium modified-methacrylate, acrylamide, caprolactone, lactide, and valeronolactone.

13. The construct of claim 8, wherein each of the plurality of hydrophobic domains or the cationic charge domains comprise a 1H-indole group; a nitrogen containing mono- or multicyclic aromatic or non-aromatic compounds.

14. The construct of claim 1, wherein the plurality of cationic charge domains comprise a primary amine, a secondary amine, a tertiary amine, a quaternary amine, a complex amino group, or an ionizable amines.

15. The construct of claim 14, wherein the plurality of cationic charge domains comprise metformin group, a morpholine group, a piperazine group, a pyridine group, a pyrrolidine group, piperidine, a thiomorpholine, a thiomorpholine oxide, a thiomorpholine dioxide, imidazole, guanidine group or creatine.

16. The construct of claim 14, wherein each of the plurality of cationic charge domains comprise a quartenary amine.

17. The construct of claim 1, wherein each of the plurality of cationic charge domains comprise spermine or spermidine.

18. The construct of claim 1 any one of the foregoing claims, wherein the at least one cargo domain comprises: RNA, DNA, ASO, PMO, PNA, mRNA, DNA vectors, oligonucleotide derivatives, a small molecule therapeutic, a peptide, a protein and/or combinations with any of these cargos as a single stranded and/or double stranded molecule.

19-20. (canceled)

21. The construct of claim 1, wherein the plurality of second ends of the dendrimer comprise spermine groups, wherein nitrogen containing groups of spermine are masked with hydrophilic masking groups.

22. The construct of claim 1, wherein the at least one cargo domain is linked to the first end of the dendrimer by a covalent bond, by hydrogen bonds, or by electrostatic attraction.

23-24. (canceled)

25. The construct of claim 4, wherein the hydrophilic masking groups are configured to be removed by esterases, glycosidases, lipases, proteases, phospholipases, phosphatases, sulfatases or the low pH in endosomes or lysosomes.

26-27. (canceled)

28. A hydrophilic masked cationic charged dendrimer comprising:
a dendrimer linked or complexed to cargo molecule(s);
hydrophilic masking groups linked to nitrogen containing groups on the termini of the dendrimer;
wherein the hydrophilic masking groups are configured to be removed by the action of enzymes or low pH found in lysosomes and/or endosomes,
wherein removal of the hydrophilic masking groups reveals the nitrogen containing groups that have cationic charges, and
wherein the cationic charged nitrogen containing groups of the dendrimer are configured to disrupt the membrane of lysosomes and/or endosomes leading to the intracellular release of the cargo molecule(s).

29-45. (canceled)

46. A hydrophilic masked cationic charged lipid nanoparticle (LNP) comprising:
a lipid nanoparticle comprises or is loaded with cargo molecule(s);
wherein the lipid nanoparticle comprises lipids that have nitrogen containing head groups that are linked to hydrophilic masking groups;
wherein the hydrophilic masking groups are configured to be removed by the action of enzymes found in lysosomes and/or endosomes,
wherein removal of the hydrophilic masking groups reveals the nitrogen containing head groups of the lipids that are ionizable or have cationic charges, and
wherein the ionizable or cationic charged head groups of the lipids are configured to disrupt the membrane of lysosomes and/or endosomes leading to the intracellular release of the cargo molecule(s).

47-58. (canceled)
